(19) United States
(12) Patent Application Publication
(10) Pub. No.: US 2014/0243419 A1
(43) Pub. Date: Aug. 28, 2014

(54) MUCOSALLY-APPLIED AGENT FOR PREVENTION, AMELIORATION OR TREATMENT OF RETINAL DISEASE

(71) Applicant: ROHTO PHARMACEUTICAL CO., LTD., Osaka (JP)

(72) Inventors: Takahiro KUROSE, Osaka (JP); Yoshihiro TAKAI, Osaka (JP); Takayuki MIYANO, Osaka (JP); Yusuke TAKEUCHI, Osaka (JP)

(73) Assignee: ROHTO PHARMACEUTICAL CO., LTD., Osaka (JP)

(21) Appl. No.: 14/267,221

(22) Filed: May 1, 2014

Related U.S. Application Data
(63) Continuation of application No. 14/182,714, filed on Feb. 18, 2014.
(60) Provisional application No. 61/766,277, filed on Feb. 19, 2013.

(30) Foreign Application Priority Data
Aug. 26, 2013 (JP) 2013-175075

(51) Int. Cl. A61K 31/121

(52) U.S. Cl. CPC A61K 31/121
USPC 514/675

(57) ABSTRACT

Application of a pharmaceutical preparation comprising geranylgeranylacetone to the mucosa of, for example, the eye, nose, oral cavity or pharynx can efficiently prevent, ameliorate or treat retinal diseases such as glaucoma, retinitis pigmentosa, age-related macular degeneration, and diabetic retinopathy.

### FIELD OF THE INVENTION

The present invention relates to a mucosally applied preparation for the prevention, amelioration or treatment of a retinal disease.

### STATE OF THE ART

Geranylgeranylacetone, in particular a mixture containing (5E,9E,13E)-geranylgeranylacetone and (5Z,9E,13E)-geranylgeranylacetone at a weight ratio of 3:2 is widely used as an oral therapeutic drug for gastric ulcer.

The use of geranylgeranylacetone with an unknown cis-trans isomer ratio (Eisai Co., Ltd.) in the ophthalmic field has been proposed. For example, the use of geranylgeranylacetone as an active ingredient of a therapeutic drug for a retinal disease has been proposed.

For example, WO 2007/014323 teaches a method for ameliorating an ocular disease such as diabetic retinopathy and glaucoma in a patient, the method comprising administering geranylgeranylacetone to the patient to increase the expression or activity of a heat shock protein in an ocular tissue, and recruiting a stem cell to the ocular tissue, thereby ameliorating the ocular disease.

“The American Journal of Pathology, Vol. 178, No. 3, March 2011, 1080-1090” teaches that intraperitoneal administration of geranylgeranylacetone to a retinal detachment-induced animal induced the expression of heat shock protein 70 and subsequently reduced the apoptosis of photoreceptor cells significantly.

“Investigative Ophthalmology & Visual Science, May 2003, Vol. 44, No. 5, 1982-1992” teaches that intraperitoneal administration of geranylgeranylacetone to a glaucoma rat model induced the expression of heat shock protein 72 and subsequently reduced retinal ganglion cell death and thereby ameliorated optic nerve damage.

“The Journal of Neuroscience, Mar. 2, 2005, 25(9), 2396-2404” teaches that oral administration of geranylgeranylacetone to a mouse with photoreceptor cell damage caused by light irradiation induced thioredoxin and heat shock protein 72 in the retinal pigment epithelium. The literature also teaches that the release of thioredoxin from the retinal pigment epithelium plays a crucial role in maintaining photoreceptor cells and that geranylgeranylacetone is useful for the protection of photoreceptor cells against light damage.

“Molecular vision, 2007, 13, 1601-1607” teaches that oral administration of geranylgeranylacetone to a mouse with retinal injury induced by ischemia significantly increased the number of surviving retinal neurons and that geranylgeranylacetone is useful for the treatment of retinal degenerative diseases that involve ischemic injury.

“Neuroscience Letters, 462, 2009, 281-285” teaches that oral administration of geranylgeranylacetone to a multiple sclerosis mouse model improved the visual function, reduced the number of degenerating axons in the optic nerve, and prevented cell loss in the ganglion.

Teprenone marketed by Eisai Co., Ltd. is a mixture containing (5E,9E,13E)-geranylgeranylacetone and (5Z,9E,13E)-geranylgeranylacetone at a weight ratio of 3:2 (WO 2004/047822, JP-9-169639 A, JP Pat. No. 4621326, JP-2006-89393 A, the Japanese Pharmacopoeia, Sixteenth Edition, and the package insert of Selbex). Hence the geranylgeranylacetone described in all the above literature is also a mixture containing (5E,9E,13E)-geranylgeranylacetone and (5Z,9E,13E)-geranylgeranylacetone at a weight ratio of 3:2. Teprenone marketed by companies other than Eisai Co., Ltd. is also a mixture containing (5E,9E,13E)-geranylgeranylacetone and (5Z,9E,13E)-geranylgeranylacetone at a weight ratio of 3:2 (for example, see reagent MSDS (202-15733); Wako Pure Chemical Industries, Ltd.).

All the above literature merely suggests the ameliorating effects of systemic administration of geranylgeranylacetone on retinal diseases.

On the other hand, an eye drop comprising teprenone, which is a mixture of (5E,9E,13E)-geranylgeranylacetone and (5Z,9E,13E)-geranylgeranylacetone at a weight ratio of 3:2, has been proposed to be used as a prophylactic or therapeutic drug for dry eye, eye strain, or eye dryness (JP-8-133967 A).

JP-2000-319170 A also discloses a clear eye drop consisting of teprenone, a phospholipid, a synthetic surfactant and water.

Conventional eye drops such as eye drops for the treatment of dry eye are formulated with the intention of delivering the drug effects to the anterior segment of the eye including the cornea. Generally, for delivery of drugs from the cornea to the anterior chamber, the tight junctions in the corneal epithelium function as a barrier. However, many eye drops are widely available which are effective for diseases in the anterior segment of the eye, in particular diseases requiring treatment of the corneal surface and its vicinity.

Therapeutic drugs for glaucoma are intended to exert therapeutic effects on the posterior segment of the eye including the retina and optic disc, but the action of the therapeutic drugs on the posterior segment of the eye is indirect. The therapeutic drugs penetrate into the anterior chamber and act on the anterior segment of the eye including the trabecular meshwork and the ciliary body to reduce intraocular pressure, thereby indirectly acting on the posterior segment of the eye.

Drugs administered as eye drops are very difficult to deliver to the posterior segment of the eye including the retina. This is because of the following reasons: the tight junctions in the corneal epithelium function as a barrier; drugs hardly diffuse from the anterior chamber to the vitreous body and the choroid; and due to the existence of a barrier called blood-retinal barrier in the retinal blood vessels, drugs absorbed from the conjunctiva etc. into the blood hardly penetrate from the blood into the retina.

Therefore, serious diseases in the posterior segment of the eye are conventionally treated mainly with intravitreal injection of drugs for delivery of the drugs to the lesion in the posterior segment of the eye.

### SUMMARY OF THE INVENTION

An object of the present invention is to provide a mucosal administration method capable of practically sufficiently preventing, ameliorating or treating a retinal disease.

### Solution to Problem

The inventors conducted extensive studies in order to solve the above problems and unexpectedly found that the administration of geranylgeranylacetone to the mucosa of, for example, the eye, nose or oral cavity can achieve excellent penetration of geranylgeranylacetone into the retina, thereby efficiently preventing, ameliorating or treating various retinal diseases.

The inventors also found that geranylgeranylacetone contained in a pharmaceutical preparation reduces the variability in the amount of the preparation dispensed or sprayed from an eye drop container or a nasal drop container and increases the flight distance of the pharmaceutical preparation sprayed from a nasal drop container.

The inventors also found that, when the pharmaceutical preparation of the present invention is stored in a container capable of preventing the transmission of light with a wavelength of 245 to 255 nm and/or light with a wavelength of 300 to 330 nm, the decomposition of GGA is efficiently inhibited.

The present invention has been completed based on the above findings and includes a method for preventing, ameliorating or treating a retinal disease, as described below.

(1) A method for preventing, ameliorating or treating a retinal disease, the method comprising the step of applying geranylgeranylacetone to the mucosa of a patient with a retinal disease.
(2) The method according to the above (1), wherein the mucosa is eye mucosa, nasal mucosa, oral cavity mucosa or pharyngeal mucosa.
(3) The method according to the above (1), comprising applying a pharmaceutical preparation comprising geranylgeranylacetone to the mucosa of a patient with a retinal disease, the pharmaceutical preparation being an eye drop, an eye wash, a contact lens-wearing solution, an eye ointment, a nasal drop, a nasal douche, a nasal ointment, an ear drop, an ear ointment, an oropharyngeal ointment, a sublingual tablet, a buccal preparation, an oral-mucosa patch or an inhalant.
(4) The method according to the above (1), wherein the retinal disease is at least one disease selected from the group consisting of glaucoma, retinitis pigmentosa, age-related macular degeneration, diabetic retinopathy, retinal detachment, diabetic maculopathy, hypertensive retinopathy, retinal vascular occlusion, retinal arteriosclerosis, retinal tear, retinal hole, macular hole, ophthalmorrhagia, posterior vitreous detachment, pigmented paravenous retinochoroidal atrophy, gyrate atrophy of the retina and choroid, choroideremia, crystalline retinopathy, retinitis punctata albescens, cone dystrophy, central areolar choroidal dystrophy, Doyne's honeycomb retinal dystrophy, vitelliform macular dystrophy, cystoid macular edema, occult macular dystrophy, Stargardt disease, retinoschisis, central serous chorioretinopathy, spinocerebellar ataxia type 7, familial exudative vitreoretinopathy, enhanced S-cone syndrome, angioid streaks, autosomal dominant optic atrophy, autosomal dominant drusen, acute zonal occult outer retinopathy, cancer-associated retinopathy, light damage, and ischemic retinopathy.
(5) The method according to the above (1), comprising applying, to the mucosa of a patient with a retinal disease, a pharmaceutical preparation comprising 0.00001 to 10% by weight of geranylgeranylacetone relative to the total amount of the pharmaceutical preparation.
(6) The method according to the above (1), comprising applying an aqueous or oily composition comprising geranylgeranylacetone to the mucosa of a patient with a retinal disease.
(7) The method according to the above (1), comprising applying, to the mucosa of a patient with a retinal disease, a geranylgeranylacetone-containing composition in the form of a liquid, a fluid, a gel, a semisolid or a solid.
(8) The method according to the above (1), comprising applying geranylgeranylacetone stored in a container to the mucosa of a patient with a retinal disease.
(9) The method according to the above (8), comprising applying, to the mucosa of a patient with a retinal disease, geranylgeranylacetone stored in a container having a part which satisfies the following conditions (a) and/or (b):

(a) average transmission of light with a wavelength of 245 to 255 nm is 35% or less;

(b) average transmission of light with a wavelength of 300 to 330 nm is 50% or less.

(10) The method according to the above (9), comprising applying, to the mucosa of a patient with a retinal disease, geranylgeranylacetone stored in a container having a part which satisfies the above conditions (a) and (b).
(11) The method according to the above (1), wherein the daily dose of GGA is 1 ng to 2000 mg.
(12) A method for protecting a retinal cell, the method comprising the step of applying geranylgeranylacetone to the mucosa of a patient with a retinal disease.
(13) The method according to the above (12), wherein the mucosa is eye mucosa, nasal mucosa, oral cavity mucosa or pharyngeal mucosa.
(14) The method according to the above (12), comprising applying a pharmaceutical preparation comprising geranylgeranylacetone to the mucosa of a patient with a retinal disease, the pharmaceutical preparation being an eye drop, an eye wash, a contact lens-wearing solution, an eye ointment, a nasal drop, a nasal douche, a nasal ointment, an ear drop, an ear ointment, an oropharyngeal ointment, a sublingual tablet, a buccal preparation, an oral-mucosa patch or an inhalant.
(15) The method according to the above (12), wherein the retinal cell is at least one type of cell selected from the group consisting of a retinal ganglion cell, an amacrine cell, a horizontal cell, a Muller glial cell, a bipolar cell, a retinal photoreceptor cell, and a retinal pigment epithelial cell.
(16) The method according to the above (12), comprising applying, to the mucosa of a patient with a retinal disease, a pharmaceutical preparation comprising 0.00001 to 10% by weight of geranylgeranylacetone relative to the total amount of the pharmaceutical preparation.
(17) The method according to the above (12), comprising applying an aqueous or oily composition comprising geranylgeranylacetone to the mucosa of a patient with a retinal disease.
(18) The method according to the above (12), comprising applying, to the mucosa of a patient with a retinal disease, a geranylgeranylacetone-containing composition in the form of a liquid, a fluid, a gel, a semisolid or a solid.
(19) The method according to the above (12), comprising applying geranylgeranylacetone stored in a container to the mucosa of a patient with a retinal disease.
(20) The method according to the above (19), comprising applying, to the mucosa of a patient with a retinal disease, geranylgeranylacetone stored in a container having a part which satisfies the following conditions (a) and/or (b):

(a) average transmission of light with a wavelength of 245 to 255 nm is 35% or less;

(b) average transmission of light with a wavelength of 300 to 330 nm is 50% or less.

(21) The method according to the above (20), comprising applying, to the mucosa of a patient with a retinal disease, geranylgeranylacetone stored in a container having a part which satisfies the above conditions (a) and (b).
(22) The method according to the above (12), wherein the daily dose of GGA is 1 ng to 2000 mg.
(23) A method for inhibiting the degeneration, impairment or destruction of a retinal cell, the method comprising the step of applying geranylgeranylacetone to the mucosa of a patient with a retinal disease.
(24) The method according to the above (23), wherein the mucosa is eye mucosa, nasal mucosa, oral cavity mucosa or pharyngeal mucosa.
(25) The method according to the above (23), comprising applying a pharmaceutical preparation comprising geranylgeranylacetone to the mucosa of a patient with a retinal disease, the pharmaceutical preparation being an eye drop, an eye wash, a contact lens-wearing solution, an eye ointment, a nasal drop, a nasal douche, a nasal ointment, an ear drop, an ear ointment, an oropharyngeal ointment, a sublingual tablet, a buccal preparation, an oral-mucosa patch or an inhalant.
(26) The method according to the above (23), wherein the retinal cell is at least one type of cell selected from the group consisting of a retinal ganglion cell, an amacrine cell, a horizontal cell, a Muller glial cell, a bipolar cell, a retinal photoreceptor cell, and a retinal pigment epithelial cell.
(27) The method according to the above (23), comprising applying, to the mucosa of a patient with a retinal disease, a pharmaceutical preparation comprising 0.00001 to 10% by weight of geranylgeranylacetone relative to the total amount of the pharmaceutical preparation.
(28) The method according to the above (23), comprising applying an aqueous or oily composition comprising geranylgeranylacetone to the mucosa of a patient with a retinal disease.
(29) The method according to the above (23), comprising applying, to the mucosa of a patient with a retinal disease, a geranylgeranylacetone-containing composition in the form of a liquid, a fluid, a gel, a semisolid or a solid.
(30) The method according to the above (23), comprising applying geranylgeranylacetone stored in a container to the mucosa of a patient with a retinal disease.
(31) The method according to the above (30), comprising applying, to the mucosa of a patient with a retinal disease, geranylgeranylacetone stored in a container having a part which satisfies the following conditions (a) and/or (b):

(a) average transmission of light with a wavelength of 245 to 255 nm is 35% or less;

(b) average transmission of light with a wavelength of 300 to 330 nm is 50% or less.

(32) The method according to the above (31), comprising applying, to the mucosa of a patient with a retinal disease, geranylgeranylacetone stored in a container having a part which satisfies the above conditions (a) and (b).
(33) The method according to the above (23), wherein the daily dose of GGA is 1 ng to 2000 mg.

Generally it is very difficult to deliver an effective amount of an active ingredient of an eye drop into the retina. For example, drugs like dry eye therapeutic drugs targeted to the anterior segment of the eye such as the cornea are known to hardly penetrate into the posterior segment of the eye. Usually drugs targeted to the retina are not administered to the nasal mucosa or oral cavity mucosa.

In contrast to these circumstances, the mucosally applied preparation of the present invention comprising geranylgeranylacetone (hereinafter sometimes abbreviated to “GGA”) is excellent in permeation of GGA through the mucosal cells of, for example, the eye, nose or oral cavity and is therefore capable of efficiently delivering GGA to the retina through mucosal application. Unlike oral drugs, injections, etc., a mucosally applied drug is an excellent form of pharmaceutical preparation in that systemic circulation of the drug is prevented and that the route of administration of the drug is non-invasive.

In addition, GGA is a medicinal substance which has been widely used and of which safety has been established.

Therefore, the preparation of the present invention is particularly useful for the treatment of diseases which usually require long-term medication, for example, slowly progressive retinal diseases including glaucoma and can be safely used for these diseases.

Conventional therapeutic drugs for retinal diseases indirectly protect retinal cells. For example, conventional therapeutic drugs for retinal diseases reduce the intraocular pressure by controlling the amount of the aqueous humor, thereby inhibiting the death of retinal nerve cells caused by the elevation of the intraocular pressure. In contrast, the mucosally applied preparation of the present invention can fundamentally prevent, ameliorate or treat a retinal disease. That is because the GGA contained in the preparation of the present invention directly inhibits the death of retinal cells and promotes the outgrowth of retinal neurites, thereby improving the cellular functions. In addition, application of the preparation of the present invention to the mucosa allows very high penetration of GGA into the retina, which further improves the effect of preventing, ameliorating or treating a retinal disease. Therefore, the mucosally applied preparation of the present invention is very useful as a prophylactic, ameliorating or therapeutic drug for a retinal disease.

GGA is a poorly water-soluble compound and thus it is very difficult to prepare a clear pharmaceutical preparation comprising a high concentration of GGA. However, since the mucosally applied preparation of the present invention comprising GGA as an active ingredient allows excellent penetration of GGA into the retina, it is not necessary to prepare a pharmaceutical preparation with extremely high concentration of GGA. Therefore, a clear pharmaceutical preparation is easily prepared.

GGA contained in the preparation of the present invention reduces the variability in the amount dispensed or sprayed each time from an eye drop container or a nasal drop container. Generally, an eye drop is instilled in an amount of 1 to 3 drops per administration, i.e., the dose of an eye drop is small. Therefore, reduced variability in the amount dispensed each time is advantageous for an eye drop. For the same reason as the case of an eye drop, reduced variability in the amount sprayed each time is advantageous for a nasal drop. Since the preparation of the present invention has reduced variability in the amount dispensed or sprayed each time, a predetermined dose is easily administered.

GGA contained in the preparation of the present invention increases the flight distance of the preparation sprayed from a nasal drop container. When a nasal drop is applied to the back of the nasal cavity, the drug is better absorbed. Therefore, long flight distance of the preparation sprayed from a nasal drop container is advantageous for a nasal drop.

Further, when the preparation of the present invention is stored in a container capable of preventing the transmission of light with a wavelength of 245 to 255 nm and/or light with a wavelength of 300 to 330 nm, the decomposition of GGA is efficiently inhibited. Therefore, quality control of the preparation of the present invention during commercial distribution, storage, etc. is easy.

### BRIEF DESCRIPTION OF THE DRAWINGS

FIG. 1 is charts showing the neuroprotective action of a GGA-containing eye drop on the eye of an NMDA-induced glaucoma rat model.

FIG. 2 is a chart showing the neuroprotective action of GGA on the eye of an NMDA-induced glaucoma rat model when GGA was administered by instillation into the nose or administered to the oral cavity.

FIG. 3 is a chart showing permeation of GGA through various types of cells.

FIG. 4 is a schematic drawing showing the test method for the flight distance of a sprayed nasal drop.

### DETAILED DESCRIPTION OF THE INVENTION

The present invention will be described in detail below.

The mucosally applied preparation of the present invention for the prevention, amelioration or treatment of a retinal disease comprises GGA. In particular, the preparation of the present invention can comprise GGA as an active ingredient.

### Geranylgeranylacetone

### (1) Types of Geometric Isomers

GGA has eight geometric isomers. Specifically, the eight geometric isomers are:
(5E,9E,13E)-6,10,14,18-tetramethyl-5,9,13,17-nonadecatetraen-2-one ((5E,9E,13E)-GGA) (all-trans form),
(5Z,9E,13E)-6,10,14,18-tetramethyl-5,9,13,17-nonadecatetraen-2-one ((5Z,9E,13E)-GGA) (5Z-mono-cis form),
(5Z,9Z,13E)-6,10,14,18-tetramethyl-5,9,13,17-nonadecatetraen-2-cone ((5Z,9Z,13E)-GGA) (13E-mono-trans form),
(5Z,9Z,13Z)-6,10,14,18-tetramethyl-5,9,13,17-nonadecatetraen-2-one ((5Z,9Z,13Z)-GGA) (all-cis form),
(5E,9Z,13E)-6,10,14,18-tetramethyl-5,9,13,17-nonadecatetraen-2-one ((5E,9Z,13E)-GGA) (9Z-mono-cis form),
(5E,9Z,13Z)-6,10,14,18-tetramethyl-5,9,13,17-nonadecatetraen-2-one ((5E,9Z,13Z)-GGA) (5E-mono-trans form),
(5E,9E,13Z)-6,10,14,13-tetramethyl-5,9,13,17-nonadecatetraen-2-one ((5E,9E,13Z)-GGA) (13Z-mono-cis form), and
(5Z,9E,13Z)-6,10,14,18-tetramethyl-5,9,13,17-nonadecatetraen-2-cone ((5Z,9E,13Z)-GGA) (9E-mono-trans form).

The 5Z-mono-cis form, the 9Z-mono-cis form and the 13Z-mono-cis form are sometimes collectively referred to as “the mono-cis form” herein.

In the present invention, the type of GGA is not limited and GGA may be one type or a combination of two or more types.

For more significant effects of the present application, preferred are the all-trans form: a mixture of geometric isomers of GGA, comprising 80% by weight or more of the all-trans form (in particular, a mixture comprising the all-trans form and the mono-cis form (in particular, the 5Z-mono-cis form) with the ratio of the all-trans form being 80% by weight or more); the mono-cis form; a mixture of geometric isomers of GGA, comprising 80% by weight or more of the mono-cis form (in particular, a mixture comprising the mono-cis form (in particular, the 5Z-mono-cis form) and the all-trans form with the ratio of the mono-cis form being 80% by weight or more); and the widely used mixture of geometric isomers, comprising the all-trans form and the 5Z-mono-cis form at a weight ratio of 3:2.

In the mixture of geometric isomers of GGA comprising 80% by weight or more of the all-trans form, the ratio of the all-trans form is preferably 82% by weight or more, more preferably 84% by weight or more, further more preferably 86% by weight or more, further more preferably 88% by weight or more, further more preferably 90% by weight or more, further more preferably 92% by weight or more, further more preferably 94% by weight or more, further more preferably 96% by weight or more, further more preferably 98% by weight or more. When the all-trans form is in the above range, significant effects of preventing, ameliorating or treating a retinal disease, of protecting a retinal cell and/or of inhibiting the degeneration, impairment or destruction of a retinal cell are exhibited.

In the mixture of geometric isomers of GGA comprising 80% by weight or more of the mono-cis form, the ratio of the mono-cis form is preferably 82% by weight or more, more preferably 84% by weight or more, further more preferably 86% by weight or more, further more preferably 88% by weight or more, further more preferably 90% by weight or more, further more preferably 92% by weight or more, further more preferably 94% by weight or more, further more preferably 96% by weight or more, further more preferably 98% by weight or more. When the mono-cis form is in the above range, significant effects of preventing, ameliorating or treating a retinal disease, of protecting a retinal cell and/or of inhibiting the degeneration, impairment or destruction of a retinal cell are exhibited.

### (2) Production of Geometric Isomers of GGA All-Trans Form

(5E,9E,13E)-GGA (all-trans form) is a compound represented by the following structural formula:

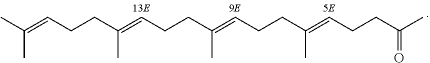 [SMILES: CC(=O)CC/C=C(\C)CC/C=C(\C)CC/C=C(\C)CCC=C(C)C]

The all-trans form can be purchased from, for example, Rionlon Development Co., Ltd.

The all-trans form can also be obtained by separating it from the 5Z-mono-cis form in a commercially available teprenone (Eisai Co., Ltd., Wako Pure Chemical Industries, Ltd., Yoshindo Inc., etc.) by, for example, silica gel chromatography using a mobile phase of n-hexane/ethyl acetate (9:1). The separation procedure of the all-trans form from the 5Z-mono-cis form in a commercially available teprenone may be outsourced to, for example, KNC Laboratories Co., Ltd.

The all-trans form can be synthesized in accordance with the method described in, for example, Bull. Korean Chem. Soc., 2009, Vol. 30, No. 9, 215-217. This literature describes, for example, the method represented by the following synthesis scheme:

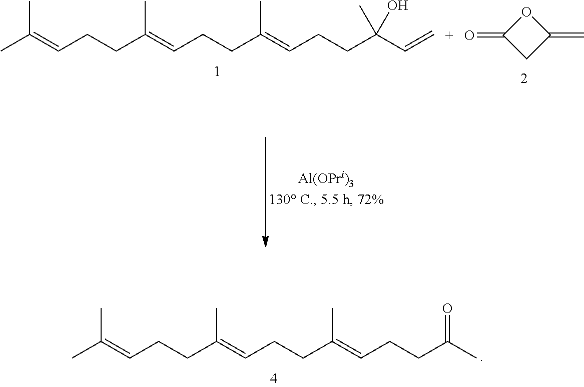

Specifically, in the above reaction formula, geranyllinalool 1 is mixed with Compound 2 and aluminum isopropoxide, and the mixture is gradually heated to 130° C. to allow the reaction to occur. After the completion of the reaction, the residual Compound 2 is removed and the reaction mixture is diluted with 5% sodium carbonate to quench the residual aluminum propoxide. In this way, the all-trans form can be obtained. The obtained all-trans form is subsequently purified by, for example, silica gel chromatography using dichloromethane as an eluent.

Mono-Cis Form

(5Z,9E,13E)-GGA (5Z-mono-cis form) is a compound represented by the following structural formula:

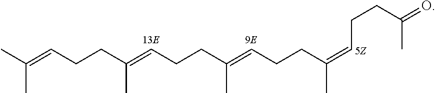 [SMILES: CC(=O)CC/C=C(/C)CC/C=C(\C)CC/C=C(\C)CCC=C(C)C]

The 5Z-mono-cis form can be obtained by separating it from a commercially available teprenone.

### Other Isomers

The other geometric isomers of GGA can be produced by a person skilled in the art by reference to the above-described methods.

A mixture of geometric isomers of GGA, for example, a mixture of the all-trans form and the 5Z-mono-cis form with the ratio of the all-trans form being over 60% by weight can be obtained by adding the all-trans form to a commercially available teprenone. In another example, a mixture of the 5Z-mono-cis form and the all-trans form with the ratio of the 5Z-mono-cis form being over 40% by weight can be obtained by adding the 5Z-mono-cis form to a commercially available teprenone.

### Amount of GGA in Pharmaceutical Preparations

The amount of GGA in the mucosally applied preparation is preferably 0.00001% by weight or more, more preferably 0.0001% by weight or more, further more preferably 0.001% by weight or more, relative to the total amount of the preparation. The amount of GGA may be 0.01% by weight or more, 0.1% by weight or more, or 1% by weight or more.

The amount of GGA in the mucosally applied preparation is preferably 95% by weight or less, more preferably 90% by weight or less, further more preferably 80% by weight or less, relative to the total amount of the preparation.

In particular, when the preparation is in a form other than a solid, for example, a liquid, a fluid, a gel or a semisolid, the amount of GGA in the preparation is preferably 10% by weight or less, more preferably 5% by weight or less, further more preferably 3% by weight or less, relative to the total amount of the preparation.

The amount of GGA in the mucosally applied preparation is, for example, about 0.00001 to 95% by weight, about 0.00001 to 90% by weight, about 0.00001 to 80% by weight, about 0.0001 to 95% by weight, about 0.0001 to 90% by weight, about 0.0001 to 80% by weight, about 0.001 to 95% by weight, about 0.001 to 90% by weight, about 0.001 to 80% by weight, about 0.01 to 95% by weight, about 0.01 to 90% by weight, about 0.01 to 80% by weight, about 0.1 to 95% by weight, about 0.1 to 90% by weight, about 0.1 to 80% by weight, about 1 to 95% by weight, about 1 to 90% by weight, or about 1 to 80% by weight, relative to the total amount of the composition.

When the mucosally applied preparation is in a form other than a solid, for example, a liquid, a fluid, a gel or a semisolid, the amount of GGA is, for example, about 0.00001 to 10% by weight, about 0.00001 to 5% by weight, about 0.00001 to 3% by weight, about 0.0001 to 10% by weight, about 0.0001 to 5% by weight, about 0.0001 to 3% by weight, about 0.001 to 10% by weight, about 0.001 to 5% by weight, about 0.001 to 3% by weight, about 0.01 to 10% by weight, about 0.01 to 5% by weight, about 0.01 to 3% by weight, about 0.1 to 10% by weight, about 0.1 to 5% by weight, about 0.1 to 3% by weight, about 1 to 10% by weight, about 1 to 5% by weight, or about 1 to 3% by weight, relative to the total amount of the composition.

When the mucosally applied preparation is in a form of a solid, the amount of GGA in the preparation is preferably 0.001 mg or more, more preferably 0.01 mg or more, further more preferably 0.1 mg or more, relative to the total amount of the preparation. The amount of GGA is preferably 1000 mg or less, more preferably 100 mg or less, further more preferably 10 mg or less.

The amount of GGA in a solid preparation is, for example, about 0.001 to 1000 mg, about 0.001 to 100 mg, about 0.001 to 10 mg, about 0.01 to 1000 mg, about 0.01 to 100 mg, about 0.01 to 10 mg, about 0.1 to 1000 mg, about 0.1 to 100 mg, or about 0.1 to 10 mg, relative to the total amount of the preparation.

When GGA is in the above range, the effects of the present application are significant, i.e., the effects of preventing, ameliorating or treating a retinal disease are sufficiently exhibited and the variability in the amount of the dispensed eye drop or the sprayed nasal drop is reduced.

### Pharmaceutical Preparations

The dosage form of the mucosally applied preparation is not particularly limited and may be any form of, for example, a liquid, a fluid, a gel, a semisolid and a solid. The mucosally applied preparation may be the one to be mixed at the time of use into a liquid, fluid, gel, semisolid or solid form. The term “semisolid” herein refers to a form having plasticity, i.e., having the property of being made into different shapes by the application of force, like an ointment.

An ophthalmic preparation of the present invention may be an aqueous composition (comprising an aqueous or hydrophilic component as a main base or carrier) or an oily composition (comprising an oily or hydrophobic component as a main base or carrier).

The amount of water in the aqueous composition is preferably 50% by weight or more, more preferably 75% by weight or more, further more preferably 90% by weight or more, relative to the total amount of the composition. The base or carrier may consist of water.

The amount of water in the oily composition is preferably less than 50% by weight, more preferably 30% by weight or less, further more preferably 20% by weight or less, relative to the total amount of the composition.

Examples of the mucosally applied preparation include ophthalmic preparations to be applied or administered to the eye mucosa (eye drops, eye washes, contact lens-wearing solutions, eye ointments (aqueous eye ointments, oil-soluble eye ointments), etc.); otorhinologic preparations to be applied or administered to the nasal mucosa or ear mucosa (for example, the preparations to be applied or administered to the nasal mucosa, including nasal drops, nasal douches, otorhinologic ointments (nasal ointments), etc., and the preparations to be applied or administered to the ear mucosa, including ear drops, otorhinologic ointments (ear ointment), etc.); oropharyngeal preparations to be applied or administered to the oral cavity mucosa or pharyngeal mucosa (for example, the preparations defined in GENERAL RULES FOR PREPARATIONS “2. Preparations for Oro-mucosal Application” in the Japanese pharmacopoeia, Sixteenth Edition, specifically including oropharyngeal solid preparations (troches, sublingual tablets, buccal preparations (buccal tablets etc.), oral cavity patches (oral-mucosa adhesive films, oral-mucosa adhesive patches, mucoadhesive tablets, etc.), medicated chewing gums, etc.), oropharyngeal semisolid preparations (oropharyngeal creams, oropharyngeal gels, oropharyngeal ointments, etc.), oropharyngeal sprays, gargles, inhalants, etc.); etc.

Among them, for easy handling by patients and excellent penetration to the retina, preferred are ophthalmic preparations, otorhinologic preparations, oral cavity preparations and pharyngeal preparations, and more preferred are eye drops and nasal drops. Preferably, the mucosally applied preparation is a preparation to be applied to the eye mucosa, nasal mucosa, oral cavity mucosa or pharyngeal mucosa.

The mucosally applied preparation can be prepared by mixing GGA with a pharmaceutically acceptable base or carrier, and optionally a pharmaceutically acceptable additive for a mucosally applied preparation and another active ingredient (a physiologically or pharmacologically active component other than GGA) in accordance with a conventional method described in, for example, Dai 16-kaisei Nihon Yakkyokuho Kaisetsusho (Commentary on the Japanese Pharmacopoeia, Sixteenth Edition).

Bases or Carriers

Examples of the base or carrier include water; an aqueous solvent such as a polar solvent; a polyalcohol; a vegetable oil; and an oily base. The base or carrier may be one kind or a combination of two or more kinds.

Additives

Examples of the additive include a surfactant, a flavor or cooling agent, an antiseptic, a bactericide or antibacterial agent, a pH adjusting agent, a tonicity agent, a chelating agent, a buffering agent, a stabilizer, an antioxidant, and a thickening agent.

The additive may be one kind or a combination of two or more kinds.

The additive will be exemplified below.

Surfactants: for example, nonionic surfactants such as polyoxyethylene (hereinafter sometimes referred to as “POE”)-polyoxypropylene (hereinafter sometimes referred to as “POP”) block copolymers (e.g., poloxamer 407, poloxamer 235, poloxamer 188), ethylenediamine POE-POP block copolymer adducts (e.g., poloxamine), POE sorbitan fatty acid esters (e.g., polysorbate 20, polysorbate 60, polysorbate 80 (TO-10 etc.)), POE hydrogenated castor oils (e.g., POE (60) hydrogenated castor oil (HCO-60 etc.)), POE castor oils, POE alkyl ethers (e.g., polyoxyethylene (9) lauryl ether, polyoxyethylene (20) polyoxypropylene (4) cetyl ether), polyoxyl stearate, etc.;

amphoteric surfactants such as glycine-type amphoteric surfactants (e.g., alkyl diaminoethyl glycine, alkyl polyaminoethyl glycine), betaine-type amphoteric surfactants (e.g., lauryldimethylaminoacetic betaine, imidazolinium betaine), etc.;

cationic surfactants such as alkyl quaternary ammonium salts (e.g., benzalkonium chloride, benzethonium chloride) etc.; etc.

The numbers in the parentheses represent the molar number of added POE or POP.

Flavors or cooling agents: for example, essential oils such as camphor, borneol, terpenes (these may be in the d-form, l-form, or dl-form), mentha water, eucalyptus oil, bergamot oil, anethole, eugenol, geraniol, menthol, limonene, mentha oil, peppermint oil, and rose oil; etc.

Antiseptics, bactericides, or antibacterial agents: for example, polidronium chloride, alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl paraoxybenzoate, ethyl paraoxybenzoate, propyl paraoxybenzoate, butyl paraoxybenzoate, oxyquinoline sulfate, phenethyl alcohol, benzyl alcohol, biguanide compounds (in particular, polyhexamethylene biguanide or its hydrochloride etc.), Glokill (Rhodia Ltd.), etc.

pH adjusting agents: for example, hydrochloric acid, sodium hydroxide, potassium hydroxide, calcium hydroxide, magnesium hydroxide, triethanolamine, monoethanolamine, diisopropanolamine, sulfuric acid, phosphoric acid, etc.

Tonicity agents: for example, sodium bisulfite, sodium sulfite, potassium chloride, calcium chloride, sodium chloride, magnesium chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, glycerin, propylene glycol, etc.

Chelating agents: for example, ascorbic acid, edetic acid tetrasodium, sodium edetate, citric acid, etc.

Buffering agents: for example, phosphate buffering agents; citrate buffering agents such as citric acid and sodium citrate; acetate buffering agents such as acetic acid, potassium acetate, and sodium acetate; carbonate buffering agents such as sodium bicarbonate and sodium carbonate; borate buffering agents such as boric acid and borax; amino acid buffering agents such as taurine, aspartic acid and its salts (e.g., potassium salts etc.), and ε-aminocaproic acid; etc.

Among the above, phosphate buffering agents are preferably used to adjust the pH of the preparation. When a phosphate buffering agent is used, adsorption of GGA to a container wall is prevented and consequently the loss of the GGA content of an eye drop is prevented. Further, other effects can also be obtained, such as prevention of clouding of the preparation during storage at low temperature, prevention of adsorption of GGA to a contact lens, and improvement of the thermal and light stabilities.

Stabilizers: for example, trometamol, sodium formaldehyde sulfoxylate (rongalit), tocopherol, sodium pyrosulfite, monoethanolamine, aluminum monostearate, glyceryl monostearate, etc.

Antioxidants: for example, water-soluble antioxidants such as ascorbic acid, ascorbic acid derivatives (ascorbic acid-2-sulfate disodium salt, sodium ascorbate, ascorbic acid-2-phosphoric acid magnesium salt, ascorbic acid-2-phosphoric acid sodium salt, etc.), sodium bisulfite, sodium sulfite, sodium thiosulfate, etc.

The mucosally applied preparation may comprise a fat-soluble antioxidant. When a fat-soluble antioxidant is contained, adsorption of GGA to a container wall is prevented and consequently the loss of the GGA content of the mucosally applied preparation is prevented. Further, adsorption of GGA to a contact lens is prevented and the thermal and light stabilities of GGA are improved.

Examples of the fat-soluble antioxidant include butyl group-containing phenols such as butylhydroxytoluene (BHT) and butylhydroxyanisole (BHA); nordihydroguaiaretic acid (NDGA); ascorbic acid esters such as ascorbyl palmitate, ascorbyl stearate, ascorbyl aminopropyl phosphate, ascorbyl tocopherol phosphate, ascorbic acid triphosphate, and ascorbyl palmitate phosphate; tocopherols such as α-tocopherol, β-tocopherol, γ-tocopherol, and δ-tocopherol; tocopherol derivatives such as tocopherol acetate, tocopherol nicotinate, and tocopherol succinate; gallic acid esters such as ethyl gallate, propyl gallate, octyl gallate, and dodecyl gallate; propyl gallate; 3-butyl-4-hydroxyquinolin-2-one; vegetable oils such as soybean oil, rapeseed oil, olive oil, and sesame oil; carotenoids such as lutein and astaxanthin; polyphenols such as anthocyanins, catechin, tannin, and curcumin; the vitamin A group including retinol, retinol esters (retinol acetate, retinol propionate, retinol butyrate, retinol octylate, retinol laurate, retinol stearate, retinol myristate, retinol oleate, retinol linolenate, retinol linoleate, retinol palmitate, etc.), retinal, retinal esters (retinal acetate, retinal propionate, retinal palmitate, etc.), retinoic acid, retinoic acid esters (methyl retinoate, ethyl retinoate, retinol retinoate, tocopheryl retinoate, etc.), dehydro forms of retinol, dehydro forms of retinal, dehydro forms of retinoic acid, provitamin A (α-carotene, β-carotene, γ-carotene, δ-carotene, lycopene, zeaxanthin, β-cryptoxanthin, echinenone, etc.), and vitamin A; CoQ10; etc. These compounds are commercially available.

Thickening agents: for example, guar gum; hydroxypropyl guar gum; high molecular cellulose compounds such as methylcellulose, ethylcellulose, hydroxypropyl methylcellulose, hydroxyethyl cellulose, and carboxymethyl cellulose sodium; gum arabic; karaya gum; xanthan gum; agar-agar; alginic acid; α-cyclodextrin; dextrin; dextran; heparin; heparinoid; heparin sulfate; heparan sulfate; hyaluronic acid; hyaluronates (sodium salts etc.); sodium chondroitin sulfate; starch; chitin and its derivatives; chitosan and its derivatives; carrageenan; sorbitol; high molecular polyvinyl compounds such as polyvinylpyrrolidone, polyvinyl alcohol, and polyvinyl methacrylate; carboxy vinyl polymers such as alkali metal polyacrylates (sodium salts, potassium salts, etc.), amine polyacrylates (monoethanolamine salts, diethanolamine salts, triethanolamine salts, etc.), and ammonium polyacrylates; casein; gelatin; collagen; pectin; elastin; ceramide; liquid paraffin; glycerin; polyethylene glycol; macrogol; polyethyleneimine alginates (sodium salts etc.); alginate esters (propylene glycol esters etc.); powdered tragacanth; triisopropanolamine; etc.

Other Prophylactic, Ameliorating or Therapeutic Components for Retinal Diseases

Preferably, the mucosally applied preparation comprises, in addition to GGA, a component that prevents, ameliorates or treats a retinal disease by a different mechanism of action from that of GGA. That is, the mucosally applied preparation preferably comprises a combination of GGA and another component as active ingredients for preventing, ameliorating or treating a retinal disease. The prophylactic, ameliorating or therapeutic component other than GGA may be one kind or a combination of two or more kinds.

Examples of such a combination include, but are not limited to, combinations of GGA and a prostaglandin F2α derivative, such as combinations of GGA and a prost drug (GGA and latanoprost, GGA and travoprost, GGA and tafluprost, GGA and bimatoprost, etc.) and combinations of GGA and a prostone drug (GGA and isopropyl unoprostone); combinations of GGA and a sympatholytic drug such as, combinations of GGA and a β-blocker (GGA and timolol maleate, GGA and gel-forming timolol, GGA and carteolol hydrochloride, GGA and gel-forming carteolol, etc.), combinations of GGA and a β1-blocker (GGA and betaxolol hydrochloride, etc.), combinations of GGA and an αβ-blocker (GGA and levobunolol hydrochloride, GGA and nipradilol, etc.), and combinations of GGA and an α1-blocker (GGA and bunazosin hydrochloride etc.); combinations of GGA and a parasympathomimetic drug, such as GGA and pilocarpine hydrochloride, and GGA and distigmine bromide; combinations of GGA and a sympathomimetic drug, such as GGA and epinephrine, GA and epinephrine bitartrate, GGA and dipivefrin hydrochloride, and combinations of GGA and an α2-blocker (GGA and brimonidine tartrate); combinations of GGA and a carbonic anhydrase inhibitor, such as GGA and acetazolamide, GGA and dorzolamide hydrochloride, and GGA and brinzolamide; combinations of GGA and a specific inhibitor to ROCK (Rho-associated coiled coil forming protein kinase), such as GGA and fasudil hydrochloride, GGA and Y-27632, GGA and AR-12286, GGA and INS-117548, GGA and SNJ-1656, and GGA and K-115; combinations of GGA and a calcium antagonist, such as GGA and lomerizine hydrochloride; combinations of GGA and an EP2 agonist, such as GGA and DE-117; combinations of GGA and an adenosine A2a receptor agonist, such as GGA and OPA-6566; combinations of GGA and a therapeutic drug for age-related macular degeneration, such as combinations of GGA and a VEGF aptamer (GGA and pegaptanib sodium) and combinations of GGA and a VEGF inhibitor (GGA and ranibizumab, and GGA and bevacizumab).

For a very high prophylactic, ameliorating or therapeutic effect for a retinal disease, among the above combinations preferred are combinations of GGA and a prostaglandin F2α derivative, combinations of GGA and a sympatholytic drug (especially, combinations of GGA and a β-blocker), combinations of GGA and a ROCK inhibitor, and combinations of GGA and a carbonic anhydrase inhibitor.

Other Pharmacologically or Physiologically Active Components

The mucosally applied preparation can comprise a pharmacologically or physiologically active component other than the prophylactic, ameliorating or therapeutic component for a retinal disease. Such a pharmacologically or physiologically active component may be one kind or a combination of two or more kinds.

Examples of the pharmacologically or physiologically active component include nerve growth factors, decongestants, drugs for restoring extraocular muscle function, anti-inflammatory drugs or astringent drugs, antihistamines or antiallergics, vitamins, amino acids, antibacterial drugs or bactericides, sugars, high molecular compounds, celluloses or their derivatives, local anesthetics, soothing agents, etc. These drugs will be exemplified below.

Nerve growth factors: for example, nerve growth factor (NGF), brain-derived neurotrophic factor (BDNF), glial cell line-derived neurotrophic factor (GDNF), etc.

Since nutritional factors including nerve growth factors are contained in the serum, it is possible to add patient's serum to a preparation to be used for the patient.

Decongestants: for example, α-adrenergic agonists such as epinephrine, epinephrine hydrochloride, ephedrine hydrochloride, oxymetazoline hydrochloride, tetrahydrozoline hydrochloride, naphazoline hydrochloride, phenylephrine hydrochloride, methylephedrine hydrochloride, epinephrine hydrogen tartrate, naphazoline nitrate, etc. These may be in the d-form, l-form, or dl-form.

Drugs for restoring extraocular muscle function: for example, cholinesterase inhibitors having an active center similar to that of acetylcholine, such as neostigmine methylsulfate, tropicamide, helenien, atropine sulfate, etc.

Anti-inflammatory drugs or astringent drugs: for example, zinc sulfate, zinc lactate, allantoin, ε-aminocaproic acid, indomethacin, lysozyme chloride, silver nitrate, pranoprofen, azulene sulfonate sodium, dipotassium glycyrrhizinate, diammonium glycyrrhizinate, diclofenac sodium, bromfenac sodium, berberine chloride, berberine sulfate, etc.

Antihistamines or antiallergics: for example, acitazanolast, diphenhydramine or its salts (hydrochloride etc.), chlorpheniramine maleate, ketotifen fumarate, levocabastine or its salts (hydrochloride etc.), amlexanox, ibudilast, tazanolast, tranilast, oxatomide, suplatast or its salts (tosilate etc.), sodium cromoglicate, pemirolast potassium, etc.

Vitamins: for example, retinol acetate, retinol palmitate, pyridoxine hydrochloride, flavin adenine dinucleotide sodium, pyridoxal phosphate, cyanocobalamin, panthenol, calcium pantothenate, sodium pantothenate, ascorbic acid, tocopherol acetate, tocopherol nicotinate, tocopherol succinate, tocopherol calcium succinate, ubiquinone derivatives, etc.

Amino acids: for example, aminoethylsulfonic acid (taurine), glutamic acid, creatinine, sodium aspartate, potassium aspartate, magnesium aspartate, magnesium potassium aspartate, sodium glutamate, magnesium glutamate, ε-aminocaproic acid, glycine, alanine, arginine, lysine, γ-aminobutyric acid, γ-aminovaleric acid, sodium chondroitin sulfate, etc. These may be in the d-form, l-form, or dl-form.

Antibacterial drugs or bactericides: for example, alkylpolyaminoethylglycine, chloramphenicol, sulfamethoxazole, sulfisoxazole, sulfamethoxazole sodium, sulfisoxazole diethanolamine, sulfisoxazole monoethanolamine, sulfisomezole sodium, sulfisomidine sodium, ofloxacin, norfloxacin, levofloxacin, lomefloxacin hydrochloride, acyclovir, etc.

Sugars: for example, monosaccharides, disaccharide, in particular, glucose, maltose, trehalose, sucrose, cyclodextrin, xylitol, sorbitol, mannitol, etc.

High molecular compounds: for example, alginic acid, sodium alginate, dextrin, dextran, pectin, hyaluronic acid, chondroitin sulfate, (completely or partially saponified) polyvinyl alcohol, polyvinylpyrrolidone, carboxy vinyl polymers, macrogol, pharmaceutically acceptable salts thereof, etc.

Celluloses or their derivatives: for example, ethylcellulose, hydroxyethyl cellulose, hydroxypropyl cellulose, hydroxypropyl methylcellulose, methylcellulose, carboxymethyl cellulose, carboxymethylcellulose sodium, carboxyethyl cellulose, nitrocellulose, etc.

Local anesthetics: for example, chlorobutanol, procaine hydrochloride, lidocaine hydrochloride, etc.

Soothing agents: benzalkonium chloride, procaine hydrochloride, etc.

Solid Preparations

Solid preparations of the present invention will be specifically illustrated below. Examples of the solid preparations include powders, granules, tablets, dispersible tablets, pills, capsules including soft capsules, mucosa patches (oral-mucosa patches such as oral-mucosa adhesive films etc.), etc. The tablets include troches, sublingual tablets, buccal tablets, mucoadhesive tablets, medicated chewing gums, etc. The buccal preparations may be, in addition to a tablet form, in the form of, for example, a granule, a tablet, a dispersible tablet, a pill or a soft capsule.

The solid preparation can be prepared by mixing GGA with a pharmaceutically acceptable base or carrier, and optionally a pharmaceutically acceptable additive for a mucosally applied preparation and another active ingredient (a physiologically or pharmacologically active component other than GGA) in accordance with a conventional method described in, for example, Dai 16-kaisei Nihon Yakkyokuho Kaisetsusho (Commentary on the Japanese Pharmacopoeia, Sixteenth Edition).

Examples of the base, carrier or additive include excipients such as saccharose, lactose, glucose, D-mannitol, and starch; binders such as gum arabic, gelatin, crystalline cellulose, hydroxypropyl cellulose, and methylcellulose; disintegrants such as carmellose and starch; stabilizers such as anhydrous citric acid, sodium laurate, and glycerol; lubricants such as magnesium stearate, calcium stearate, talc, and colloidal silica; sweeteners; preservatives; demulcents; diluents; buffering agents; aromatizing agents; colorants; etc. The solid preparation may be coated with gelatin, saccharose, gum arabic, carnauba wax, etc., or may be encapsulated.

The mucosa adhesive film can be prepared by dissolving GGA in a solvent such as water together with, for example, an excipient and a water dispersible polymer such as hydroxypropyl cellulose and hydroxypropyl methylcellulose, and optionally with an additive and a physiologically or pharmacologically active component other than GGA, and casting the resulting solution into a film. In order to provide moderate elasticity for the film, glycols such as polyethylene glycol may be added, and in order to increase the adhesion of the film to the oral cavity mucosa, a bioadhesive polymer such as polycarbophil and carbopol may be added.

pH

The pH of the mucosally applied preparation of the present invention is preferably 4 or higher, more preferably 5.5 or higher, further more preferably 6 or higher, further more preferably 6.5 or higher. When the pH of the preparation is in the above range, excellent thermal and light stabilities of GGA can be achieved.

The pH of the mucosally applied preparation is preferably 9 or lower, more preferably 8.5 or lower, further more preferably 8 or lower, further more preferably 7.5 or lower. When the pH of the preparation is in the above range, the preparation is less irritating to the mucosa.

### Kit

The mucosally applied preparation of the present invention may be a single composition comprising all the components. Alternatively, the mucosally applied preparation may be any type of kit comprising, for example, two or three compositions. Examples of such a kit include a kit in which GGA and a pharmacologically or physiologically active component other than GGA are contained in separate compositions; and a kit in which GGA and a particular additive are contained in separate compositions. The kit may comprise different compositions stored in separate containers. The kit may comprise compositions required to be mixed at the time of use, i.e., different compositions separately stored in a container that allows the compositions to be mixed at the time of use.

When the mucosally applied preparation of the present invention is a kit comprising a composition comprising GGA and a composition comprising another component (including the above cases of a kit comprising different compositions stored in separate containers and of a kit comprising compositions to be mixed at the time of use), the above-described amount of GGA is the ratio relative to the total amount of the mixed compositions.

### Containers

The mucosally administered preparation of the present invention is usually loaded into or stored in a container. The type of container is not particularly limited and examples thereof include a plastic container, a metal container, a glass container, etc. Examples of the container also include a container in which the whole or at least part of the surface in contact with the preparation is made of at least one material selected from the group consisting of a plastic (for example, a polyolefin, an acrylic acid resin, a terephthalic acid ester, a 2,6-naphthalene dicarboxylic acid ester, a polycarbonate, a polymethylpentene, a fluorine resin, a polyvinyl chloride, a polyamide, an ABS resin, an AS resin, a polyacetal, a modified polyphenylene ether, a polyarylate, a polysulfone, a polyimide, a cellulose acetate, or a hydrocarbon optionally substituted with a halogen atom), a metal (aluminum) and glass. The container made of the above material refers to a container comprising the above material in an amount of 50% by weight or more, preferably 60% by weight or more, further more preferably 70% by weight or more, relative to the total weight of a single container (container main body). A mixture or copolymer of materials selected from the above materials may be used.

Examples of the polyolefin include polyethylenes (including high density polyethylene, low density polyethylene, ultra low density polyethylene, linear low density polyethylene, ultra high molecular weight polyethylene, etc.), polypropylenes (including isotactic polypropylene, syndiotactic polypropylene, atactic polypropylene, etc.), ethylene-propylene copolymers, etc.

Examples of the acrylic acid resin include acrylic acid esters such as methyl acrylate, methacrylic acid esters such as methyl methacrylate, cyclohexyl methacrylate and t-butyl cyclohexyl methacrylate, etc.

Examples of the terephthalic acid ester include polyethylene terephthalate, polytrimethylene terephthalate, polybutylene terephthalate, etc.

Examples of the 2,6-naphthalene dicarboxylic acid ester include polyethylene naphthalate, polybutylene naphthalate, etc.

Examples of the fluorine resin include fluorine-substituted polyethylenes (polytetrafluoroethylene, polychlorotrifluoroethylene, etc.), polyvinylidene fluoride, polyvinyl fluoride, perfluoroalkoxy fluorine resins, tetrafluoroethylene-hexafluoropropylene copolymers, ethylene-tetrafluoroethylene copolymers, ethylene-chlorotrifluoroethylene copolymers, etc.

Examples of the polyamide include nylon etc.

Examples of the polyacetal include polyacetals consisting of oxymethylene units, polyacetals containing oxyethylene units, etc.

Examples of the modified polyphenylene ether include polystyrene-modified polyphenylene ether etc.

Examples of the polyarylate include amorphous polyarylate etc.

Examples of the polyimide include aromatic polyimides such as the one obtained by polymerizing pyromellitic dianhydride and 4,4′-diaminodiphenyl ether.

Examples of the cellulose acetate include cellulose diacetate, cellulose triacetate, etc.

Examples of the hydrocarbon optionally substituted with a halogen atom include hydrocarbons such as methane, ethane, propane, butane, ethylene, propylene, 1-butene, 2-butene and 1,3-butadiene; hydrocarbons substituted with a fluorine atom such as fluoromethane, difluoromethane, fluoroform, tetrafluoromethane, 1,1-difluoroethane, 1,2-difluoroethane, 1-fluoropropane, 2-fluoropropane, 1,2-fluoropropane, 1,3-fluoropropane, 1-fluorobutane, 2-fluorobutane, vinyl fluoride, 1,1-difluoroethylene, 1,2-difluoroethylene, trifluoroethylene, tetrafluoroethylene, 3-fluoropropene, 1,3-fluoropropene, 1,1,4,4-tetrafluorobutadiene and perfluorobutadiene; hydrocarbons substituted with a chlorine atom such as chloromethane, dichloromethane, chloroform, tetrachloromethane, 1,1-dichloroethane, 1,2-dichloroethane, 1-chloropropane, 2-chloropropane, 1,2-chloropropane, 1,3-chloropropane, 1-chlorobutane, 2-chlorobutane, vinyl chloride, 1,1-dichloroethylene, 1,2-dichloroethylene, trichloroethylene, tetrachloroethylene, 3-chloropropene, 1,3-chloropropene, 1,1,4,4-tetrachlorobutadiene and perchlorobutadiene; hydrocarbons substituted with a bromine atom such as bromomethane, dibromomethane, bromoform, tetrabromomethane, 1,1-dibromoethane, 1,2-dibromoethane, 1-bromopropane, 2-bromopropane, 1,2-bromopropane, 1,3-bromopropane, 1-bromobutane, 2-bromobutane, vinyl bromide, 1,1-dibromoethylene, 1,2-dibromoethylene, tribromoethylene, tetrabromoethylene, 3-bromopropene, 1,3-bromopropene, 1,1,4,4-tetrabromobutadiene and perbromobutadiene; hydrocarbons substituted with an iodine atom such as iodomethane, diiodomethane, iodoform, tetraiodomethane, 1,1-diiodoethane, 1,2-diiodoethane, 1-iodopropane, 2-iodopropane, 1,2-iodopropane, 1,3-iodopropane, 1-iodobutane, 2-iodobutane, vinyl iodide, 1,1-diiodoethylene, 1,2-diiodoethylene, triiodoethylene, tetraiodoethylene, 3-iodopropene, 1,3-iodopropene, 1,1,4,4-tetraiodobutadiene and periodobutadiene; etc.

Preferably, the material of the container is at least one material selected from the group consisting of a terephthalic acid ester (in particular polyethylene terephthalate), a polycarbonate, a polymethylpentene, a fluorine-substituted polyethylene (in particular polytetrafluoroethylene), a 2,6-naphthalene dicarboxylic acid ester (in particular polyethylene naphthalate, and polybutylene naphthalate), a polyolefin (in particular polyethylene, and polypropylene), and a methacrylic acid ester (in particular methyl methacrylate).

The container may have a layer or film made of the above material on the inner surface. Alternatively, the container itself may be made of the above material. It is only necessary that at least part of the surface in contact with the preparation is made of the above material, and preferably the whole surface in contact with the preparation is made of the above material.

The container may be produced by integral molding or constituted of two or more parts. In cases where the container is constituted of two or more parts, only one part or two or more parts may be made of the above material, and each of the parts may be made of a different type of the above material. In the case where the container has a spout or nozzle, like an eye drop container, an eye wash container, a nasal drop container or an ointment container, the whole of the container including the spout or nozzle may be made of the above material, or only the main body excluding the spout or nozzle may be made of the above material. The whole inner surface of the container may have a layer or film made of the above material. Only the inner surface of the main body may have a layer or film made of the above material.

The shape, volume, wall thickness, etc. of the container are not particularly limited. The shape, volume, wall thickness, etc. are selected from those usually employed, depending on the type of the container.

In cases where the inner wall of the container has a layer or film made of the above material, the layer or film may be stacked on the main body of the container or formed thereon by vapor deposition, plasma CVD, plasma polymerization, sputtering, etc. The thickness of the layer or film made of the above material is not particularly limited and may be, for example, about 10 nm to 5 mm.

The weight of the mucosally applied preparation of the present invention stored in a single container is not particularly limited but is preferably about 0.0001 to 100 g, more preferably about 0.001 to 50 g, further more preferably about 0.01 to 30 g.

The mucosally administered preparation of the present invention is preferably stored in a container having a part which satisfies the following conditions (a) and/or (b), more preferably having a part which satisfies both of the following conditions (a) and (b) and thereby the decomposition of GGA is efficiently inhibited:

(a) average transmission of light with a wavelength of 245 to 255 nm is 35% or less, especially 33% or less, or even 30% or less;

(b) average transmission of light with a wavelength of 300 to 330 nm is 50% or less, especially 45% or less, or even 40% or less.

It is only necessary that at least part of the container has the above average transmission value(s). Such a part having the above average transmission value(s) preferably covers 1% or more of the total area of the container wall, especially preferably 5% or more of the total area, especially preferably 10% or more of the total area, especially preferably 30% or more of the total area, especially preferably 50% or more of the total area. When the preparation of the present invention is stored in such a container, the decomposition of GGA is efficiently inhibited.

The transmission through the container is measured by cutting out a piece from the container in a size sufficient to cover the light path of a spectrometer and measuring the transmission through the cut piece.

The GGA-containing mucosally applied preparation stored in a container which satisfies the above condition (a), preferably satisfies both of the above conditions (a) and (b), is not limited to the preparation for the prevention, amelioration or treatment of a retinal disease.

### Target Diseases

A target retinal disease of the present invention may be any retinal disease as long as it is a disease involving the degeneration, impairment or destruction of cells constituting the retina, or a disease resulting from the degeneration, impairment or destruction of cells constituting the retina. Examples of such diseases include glaucoma, retinitis pigmentosa, age-related macular degeneration, diabetic retinopathy, retinal detachment, diabetic maculopathy, hypertensive retinopathy, retinal vascular occlusion (retinal artery occlusion; retinal vein occlusion such as central retinal vein occlusion and branch retinal vein occlusion; etc.), retinal arteriosclerosis, retinal tear, retinal hole, macular hole, ophthalmorrhagia, posterior vitreous detachment, pigmented paravenous retinochoroidal atrophy, gyrate atrophy of the retina and choroid, choroideremia, crystalline retinopathy, retinitis punctata albescens, cone dystrophy, central areolar choroidal dystrophy, Doyne's honeycomb retinal dystrophy, vitelliform macular dystrophy, cystoid macular edema, occult macular dystrophy, Stargardt disease, retinoschisis, central serous chorioretinopathy (central retinopathy), spinocerebellar ataxia type 7, familial exudative vitreoretinopathy, enhanced S-cone syndrome, angioid streaks, autosomal dominant optic atrophy, autosomal dominant drusen, familial drusen, acute zonal occult outer retinopathy, cancer-associated retinopathy, light damage, ischemic retinopathy, etc.

Among these, suitable target diseases are glaucoma, retinitis pigmentosa, age-related macular degeneration, and diabetic retinopathy, and more suitable is glaucoma.

The target disease of the present invention may involve or result from the impairment of any type of cells constituting the retina. Examples of the cells constituting the retina include retinal ganglion cells, amacrine cells, horizontal cells, Muller glial cells, bipolar cells, retinal photoreceptor cells (cones and rods), retinal pigment epithelial cells, etc. Particularly suitable is a disease involving or resulting from the impairment of retinal ganglion cells or retinal pigment epithelial cells.

The target disease of the present invention may involve or result from the impairment of any of the layers constituting the retina, i.e., the inner limiting membrane, the nerve fiber layer, the ganglion cell layer, the inner plexiform layer, the inner nuclear layer, the outer plexiform layer, the outer nuclear layer, the external limiting membrane, the photoreceptor cell layer, and the retinal pigment epithelium layer. Particularly suitable is a disease involving or resulting from the impairment of the ganglion cell layer, the inner nuclear layer, or the outer nuclear layer.

The target disease may be one retinal disease or two or more retinal diseases.

The target patient to whom the present invention is applied is preferably a patient with the above retinal, disease(s).

As described above, the GGA contained in the mucosally applied preparation of the present invention exhibits a protective action on retinal cells. That is, GGA promotes the survival of retinal cells or inhibits the degeneration, impairment or destruction of retinal cells, thereby preventing, ameliorating or treating a retinal disease. Therefore, the mucosally applied preparation of the present invention can be used as a GGA-containing mucosally administered preparation for protecting a retinal cell, for promoting the survival of a retinal cell, or for inhibiting the degeneration, impairment or destruction of a retinal cell. Since GGA has the action of inducing the neurite outgrowth of cells such as retinal cells, the mucosally applied preparation of the present invention can also be used as a GGA-containing mucosally applied preparation for promoting or inducing the outgrowth of (retinal) neurites, for improving (retinal) cellular functions, for enhancing or improving the viability of a retinal cell.

Suitable target retinal cells for these preparations are the same as those listed above. The components and their amounts in the preparations, the dosage forms of the preparations, etc. are the same as those described for the above mucosally applied preparation of the present invention for the prevention, amelioration or treatment of a retinal disease.

In the present invention, the term “prevention” is understood to include prevention of or delay in the onset of a disease or reduction in the incidence of a disease. The terms “amelioration” and “treatment” are understood to include reduction in the symptoms, delay in the progress of the symptoms, and cure or remission.

### Usage

The dosage of the mucosally administered preparation of the present invention is not particularly limited and is preferably administered, for example, once or more a day. The frequency of administration per day may be 20 times or less, 15 times or less, 10 times or less, 8 times or less, or 6 times or less. In another example, the frequency of administration per day may be about 1 to 20 times, about 1 to 15 times, about 1 to 10 times, about 1 to 8 times, and about 1 to 6 times. More preferred dosage and route of administration, which vary depending on the type of the pharmaceutical preparation, are as follows.

When the preparation of the present invention is an eye drop, the eye drop comprising GGA in the above concentration range is instilled, for example, about 1 to 5 times a day, preferably about 1 to 3 times a day, in an amount of about 1 to 2 drops each time.

When the preparation of the present invention is an eye wash, eye washing is performed, for example, about 1 to 10 times a day, preferably about 1 to 5 times a day, each time using about 1 to 20 mL of the eye wash comprising GGA in the above concentration range.

When the preparation of the present invention is an eye ointment, the eye ointment comprising GGA in the above concentration range is applied to the eye, for example, about 1 to 5 times a day, preferably about 1 to 3 times a day, in an amount of about 0.001 to 5 g each time.

When the preparation of the present invention is a nasal drop, the nasal drop comprising GGA in the above concentration range is instilled into the nose by spraying etc., for example, about 1 to 5 times a day, preferably about 1 to 3 times a day, in an amount of about 0.0005 to 5 mL each time.

When the preparation of the present invention is a nasal douche, nasal washing is performed, for example, about 1 to 10 times a day, preferably about 1 to 5 times a day, each time using about 1 to 20 mL of the nasal douche comprising GGA in the above concentration range.

When the preparation of the present invention is an ear drop, the ear drop comprising GGA in the above concentration range is instilled into the ear, for example, about 1 to 5 times a day, preferably about 1 to 3 times a day, in an amount of about 1 to 2 drops each time.

When the preparation of the present invention is an oropharyngeal spray preparation (inhalant, spray, etc.), the preparation comprising GGA in the above concentration range is sprayed into the oropharynx, for example, about 1 to 5 times a day, preferably about 1 to 3 times a day, in an amount of about 0.0005 to 5 mL each time.

When the preparation of the present invention is a semisolid preparation (cream, gel, ointment, etc.) for the nose, ear or oropharynx, the ointment comprising GGA in the above concentration range is applied to the nose, ear or oropharynx, for example, about 1 to 5 times a day, preferably about 1 to 3 times a day, in an amount of about 0.001 to 5 g each time.

When the preparation of the present invention is an oropharyngeal solid preparation (sublingual tablet, troche, buccal tablet, mucoadhesive tablet, medicated chewing gum, etc.), the tablet comprising GGA in the above concentration range is applied to the oropharynx, for example, 1 to 10 times a day, preferably about 1 to 6 times a day.

In any of the above types of preparations, the daily dose of GGA is not particularly limited but is preferably 1 ng or more, more preferably 50 ng or more, further more preferably 500 ng or more, particularly preferably 5 μg or more. The daily dose of GGA may be 2000 mg or less, preferably 50 mg or less, more preferably 20 mg or less, further more preferably 10 mg or less.

In another example, the daily dose of GGA in any of the above types of preparations is about 1 ng to 2000 mg, about 50 ng to 50 mg, about 50 ng to 20 mg, about 50 ng to 10 mg, about 500 ng to 50 mg, about 500 ng to 20 mg, about 500 ng to 10 mg, about 5 μg to 50 mg, about 5 μg to 20 mg, or about 5 μg to 10 mg.

The administration period varies depending on the kind and stage of the disease, the age, weight, and sex of the patient, etc., and is appropriately selected from, for example, the range from about one day to 30 years. For example, when the patient has a retinal disease such as glaucoma, retinitis pigmentosa, age-related macular degeneration and diabetic retinopathy, the administration period is, for example, 3 days or more, preferably 5 days or more, more preferably 14 days or more, further more preferably 30 days or more, particularly preferably 90 days or more. Also, the administration period is, for example, 50 years or less, 30 years or less, 20 years or less, 10 years or less, or 5 years or less. In some cases, the administration of the preparation for about 1 to 20 years, especially as short as about 1 to 10 years, can prevent, ameliorate or cure a retinal disease. In cases where the progress of a retinal disease is inhibited by the administration of the mucosally applied preparation of the present invention via its retinal protective action, the administration can be further continued.

The present invention also includes a method for protecting a retinal cell, a method for promoting the survival of a retinal cell, a method for inhibiting the degeneration, impairment or destruction of a retinal cell, a method for promoting or inducing the outgrowth of (retinal) neurites, a method for improving (retinal) cellular functions, and a method for enhancing or improving the viability of a retinal cell, these methods comprising applying (instilling, pasting, spraying, placing, attaching, etc.) a mucosally applied preparation (pharmaceutical preparation or composition) comprising GGA to the mucosa of a patient with a retinal disease so that an effective amount of GGA is administered.

In these methods, GGA or the mucosally applied preparation comprising GGA (composition or pharmaceutical preparation comprising GGA) is stored in a container and can be dispensed from the container at the time of application to the mucosa of a patient with a retinal disease. The present invention further includes a method for protecting a retinal cell, a method for promoting the survival of a retinal cell, a method for inhibiting the degeneration, impairment or destruction of a retinal cell, a method for promoting or inducing the outgrowth of (retinal) neurites, a method for improving (retinal) cellular functions, and a method for enhancing or improving the viability of a retinal cell, these methods comprising the step of loading GGA or a mucosally applied preparation comprising GGA (composition or pharmaceutical preparation comprising GGA) into a container, and the step of applying, to the mucosa of a patient with a retinal disease, the GGA or the mucosally applied preparation comprising GGA (composition or pharmaceutical preparation comprising GGA) dispensed from the container. The container used in these methods is the same as illustrated for the mucosally applied preparation of the present invention.

In these methods of the present invention, the mucosally applied preparation comprising GGA is the same as the above illustrated mucosally applied preparation of the present invention. In these methods of the present invention, the route of administration and dosage, the type of retinal, disease, the type of retinal cell, the impaired retinal layer, the target patient, the definitions of “prevention”, “amelioration”, and “treatment”, etc. are the same as those described above.

### Examples

The present invention will be described in more detail below with reference to Examples, but the present invention is not limited thereto. In the following Examples, the amount of each component may be expressed in terms of w/v %. However, regarding the composition of each sample below, the amount of each component expressed in terms of w/v % is substantially the same value as the amount of each component expressed in terms of % by weight.

### (1) Preparation of Geranylgeranylacetone

Commercially available teprenone (all-trans form: 5Z-mono-cis form=3:2 (weight ratio)) (Wako Pure Chemical Industries, Ltd.) was purchased and the all-trans form was separated and purified by silica gel chromatography.

The above preparative purification was performed using silica gel (PSQ60B, Fuji Silysia Chemical Ltd.) filled into a glass tube and a mobile phase of n-hexane/ethyl acetate (9:1). After the separation, each fraction was concentrated and dried under reduced pressure, and the degree of purification and the structure of the all-trans form were determined by GC and ¹H-NMR (solvent: deuterated chloroform; internal standard: tetramethylsilane), respectively (about 20% yield).

### GC Measurement Conditions

Column: DB-1 (J&W Scientific, 0.53 mm×30 m, film thickness of 1.5 μm)
Column temperature: 200° C., rising at 5° C./min to 300° C. (10 min)
Vaporizing chamber temperature: 280° C.
Detector temperature: 280° C.
Carrier gas: helium
Hydrogen pressure: 60 kPa
Air pressure: 50 kPa
Makeup gas pressure: 75 kPa (nitrogen gas)
Total flow: 41 mL/min
Column flow: 6.52 mL/min
Linear velocity: 58.3 cm/sec
Split ratio: 5:1
Injection volume: 1 μL of 0.1 g/100 mL sample (in ethanol)

### (2) Evaluation of Protective Effect on Retinal Ganglion Cells Against NMDA-Induced Nerve Damage

In recent years the glutamate analogue NMDA (N-methyl-D-aspartate) has been widely reported to be one of causative agents of neurodegenerative diseases including Alzheimer's disease. In the ophthalmic field, NMDA is considered to be involved in optic nerve damage in glaucoma (Brain Research Bulletin, 81 (2010) 349-358). Accordingly, in this experiment rat models with glaucoma induced by NMDA were used to evaluate the neuroprotective effect of GGA.

### (2-1) Administration by Instillation to the Eye Test Method

To Sprague-Dawley (SD) rats (n=8), an eye drop containing 1% (w/v) GGA (all-trans form) or a control eye drop not containing GGA was pre-administered by instillation to the eye twice a day for five days. On the fifth day after the start of the instillation, 5 μL of 4 mM NMDA was intravitreously administered to induce nerve damage (Test 1).

In the same manner as above, to Sprague-Dawley (SD) rats (n=10), a 1% memantine solution or a base (PBS) was pre-administered by instillation to the eye twice a day for five days. On the fifth day after the start of the instillation, 5 μL of 4 mM NMDA was intravitreously administered to induce nerve damage. The 1% memantine solution was prepared by crushing MEMARY 20 mg TABLETS (Daiichi Sankyo) and suspending the drug in PBS so that the concentration of memantine was 1 w/v % (Test 2).

Three days after the administration of NMDA, the eyeballs were harvested, fixed in half Karnovsky's fixative solution for 24 hours, embedded in paraffin, thin sectioned and stained with hematoxylin-eosin (HE) to prepare histopathological sections. The histological sections were observed under an optical microscope and the thickness (μm) of the inner plexiform layer (IPL) of the retina was measured. Based on the thickness of the inner plexiform layer (IPL) of the retina, the neuroprotective effect of the tested eye drops was evaluated.

The composition of the eye drops used in Test 1 is shown in Table 1 below.

TABLE 1
Unit: g/100 mL

| | GGA-containing | Control |
|---|---|---|
| Components | eye drop | eye drop |
| GGA (all-trans form) | 1 | — |
| Boric acid | 1.3 | 1.3 |
| Borax | 0.4 | 0.4 |
| Polysorbate 80 | 2 | 2 |
| Polyoxyethylene hydrogenated | 2 | 2 |
| castor oil 60 | | |
| Polyoxyethylene castor oil | 0.1 | 0.1 |
| Dibutylhydroxytoluene (BHT) | 0.005 | 0.005 |
| Purified water | q.s. | q.s. |

### Results

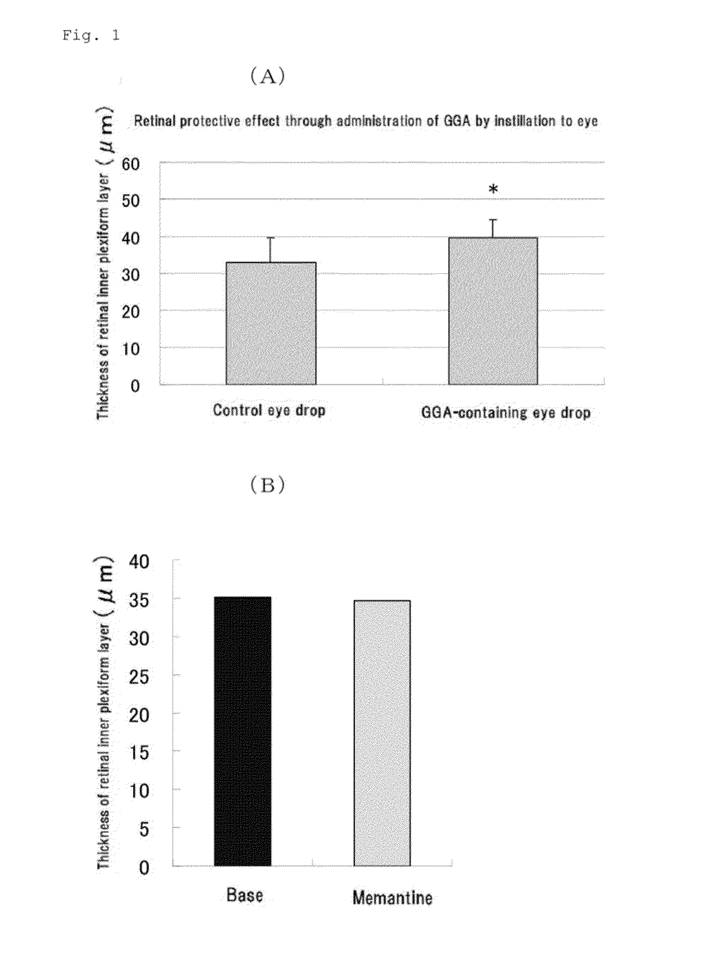

The results are shown in FIG. 1. The GGA-containing eye drop showed significant neuroprotective effect on the NMDA-induced nerve damage as compared with the control eye drop (*p<0.05, t-test) (Test 1).

On the other hand, no significant difference was observed between the memantine group and the control group and no neuroprotective effect like the effect exhibited by GGA was observed (Test 2). Memantine is an oral drug for Alzheimer therapy, but has been reported to also have a protective action on the retinal nerve. This experiment revealed that, mucosal administration (e.g., administration to the eye mucosa) of a drug which is usually orally administered for a protective action on the retina does not provide any protective action on the retina; but in contrast, mucosal administration of GGA provides a protective action on the retinal nerve.

(2-2) Administration by Instillation into the Nose and Administration to the Oral Cavity

### Test Method

To Sprague-Dawley (SD) rats (n=6), 20 μL of 100% (w/v) GGA (all-trans form) was intranasally administered once a day for five days and after each administration the rats were maintained under anesthesia for 30 minutes. On the third day after the start of the administration of GGA, 5 μL of 4 mM NMDA was intravitreously administered to induce nerve damage.

In the same manner as above, to Sprague-Dawley (SD) rats (n=6), 100 μL of 100% (w/v) GGA (all-trans form) was administered to the oral cavity once a day for five days and after each administration the rats were maintained under anesthesia for 30 minutes. On the third day after the start of the administration of GGA, 5 μL of 4 mM NMDA was intravitreously administered to induce nerve damage.

Three days after the administration of NMDA, the eyeballs were harvested, fixed in Karnovsky's fixative solution for 24 hours, embedded in paraffin, thin sectioned and stained with hematoxylin-eosin (HE) to prepare histopathological sections. The histological sections were observed under an optical microscope and the thickness (μm) of the inner plexiform layer (IPL) of the retina was measured. Based on the thickness of the inner plexiform layer (IPL) of the retina, the neuroprotective effect of the tested eye drops was evaluated.

### Results

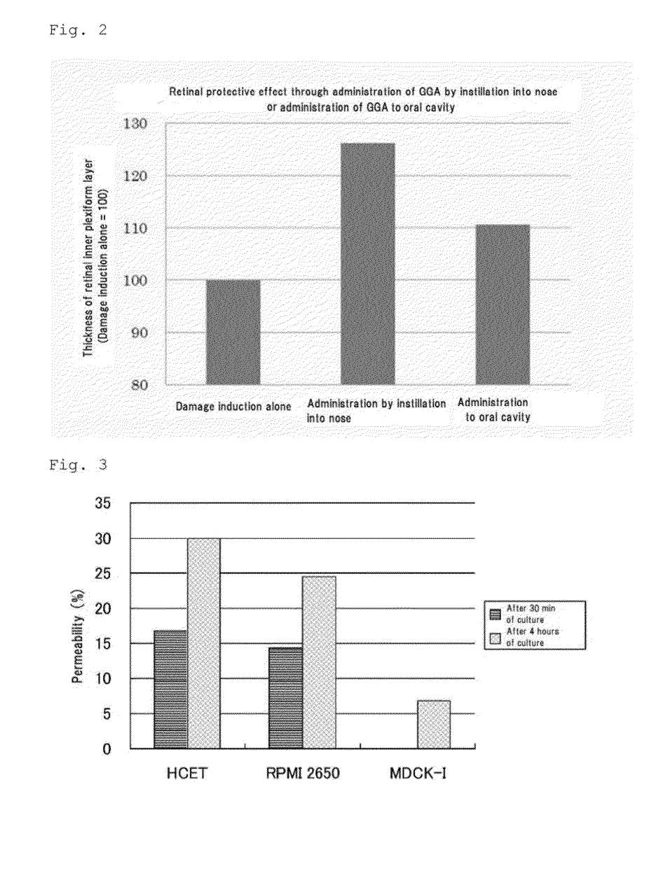

The results are shown in FIG. 2. The administration of GGA by instillation into the nose and the administration of GGA to the oral cavity showed apparent neuroprotective effect on the NMDA-induced nerve damage as compared with the case where no GGA was administered.

### (3) Evaluation of Cell Permeability

### Test Method

The following test was performed using three types of cells, human corneal epithelial cells (HCET), human nasal septum squamous carcinoma cells (RPMI-2650), and canine renal tubular epithelial cells (MDCK-I), which are often used as an intestinal permeability assay model. The medium used for culture of each type of cells is shown below.

HCET: a liquid medium containing an equal ratio of Dulbecco's modified Eagle's basal medium/Ham's F12 (DMEM/F-12, Invitrogen) supplemented with 0.5% DMSO (Wako Pure Chemical Industries, Ltd.), 10 ng/mL epidermal growth factor (R&D), 5 μg/mL insulin (Invitrogen) and 5% (v/v) fetal calf serum (Daiichi Pure Chemicals)

RPMI-2650: a minimum essential medium (MEM, Invitrogen) supplemented with 10% (v/v) fetal calf serum (Daiichi Pure Chemicals)

MDCK-I: high glucose (4.5 g/L) Dulbecco's modified Eagle's basal medium with pyruvate (DMEM, Invitrogen) supplemented with 10% (v/v) fetal calf serum (Daiichi Pure Chemicals)

GGA having a weight ratio of the all-trans form to the 5Z-mono-cis form of 6:4 (Wako Pure Chemical Industries, Ltd.) was used as a test substance. Amounts of 100 mg of GGA and 0.25 mg of α-tocopherol (Wako Pure Chemical Industries, Ltd.) as an antioxidant were weighed and dissolved in 789 mg of 100% ethanol. The GGA dissolved in ethanol was diluted and dissolved in serum-free medium appropriate for each type of cells to a final concentration of 0.1 w/v %.

Each type of cells was seeded onto a Transwell 24-well microplate (CORNING) at 5.0×10, cells per well and cultured under 5% CO₂ at 37° C. To each of the Transwells' lower chambers, 600 μL of the same medium as used for cell culture was added. After five days of culture, the culture supernatant was removed by suction. The above 0.1% GGA solution was added to each well in an amount of 100 μL and the cells were cultured under 5% CO₂ at 37° C. for 30 minutes or for 4 hours. After the culture period, the culture supernatant in the Transwells was recovered and the residual GGA in the supernatant was measured. The residual GGA ratio (%) was subtracted from 100 to give the permeability (%) of GGA through the Transwells inserts onto which the cells were seeded.

### Measurement Method for GGA Concentration

The concentration of GGA was measured as follows.

In accordance with the measurement conditions for the elution test described in PFSB/ELD Notification No. 0412007 “teprenone 100 mg/g fine granule”, the GGA concentration of each eye drop was determined from the area value of the 5Z-mono-cis form (Ac) and the area value of the all-trans form (At) using, as a reference standard, Japanese Pharmacopoeia “teprenone reference standard (all-trans form: 5Z-mono-cis form=about 6:4 (weight ratio) produced by Pharmaceutical and Medical Device Regulatory Science Society of Japan)” or teprenone (Wako Pure Chemical Industries, Ltd.) under the HPLC measurement conditions described below.

### HPLC Measurement Conditions

Detector: ultraviolet absorption spectrometer (measurement wavelength: 210 nm)
Column: YMC-Pack ODS-A (inner diameter: 4.6 mm, length: 15 cm, particle diameter: 3 μm)
Column temperature: 30° C.
Mobile phase: 90% acetonitrile solution
Flow rate: 1.2 to 1.3 mL/min (The 5Z-mono-cis form and the all-trans form are eluted in this order.)
Injection volume: 5 μL of 0.05 g/100 mL sample

### Results

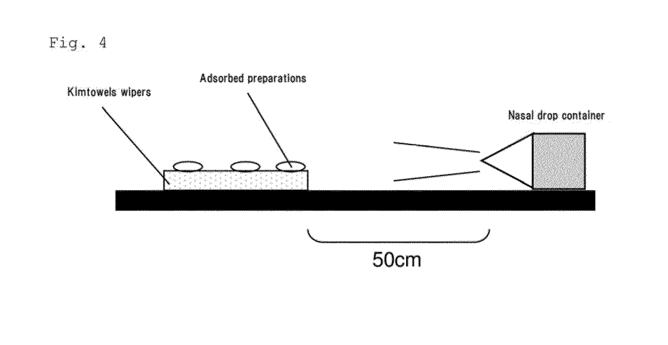

The results are shown in FIG. 3. The permeability of GGA through HCET and RPMI-2650 was higher than that through MDCK-I. These results revealed that GGA more easily permeates through the cornea and the nasal mucosa than through the intestinal mucosa and therefore administration by instillation to the eye or into the nose can deliver more GGA to the retinal tissue as compared with the oral administration.

(4) Evaluation of the Flight Distance from Nasal Drop Container

Test preparations whose composition is shown in Table 2 were prepared using teprenone (Wako Pure Chemical Industries, Ltd.). The test preparations of Examples were prepared as follows. To surfactants (polysorbate 80 and POE castor oil) warmed to 65° C., teprenone was added and dissolved under stirring in hot water bath at 65° C. for 2 minutes. Water at 65° C. was added, a buffer solution containing uranin (Wako Pure Chemical. Industries, Ltd., manufacturer's product code: 216-00102) and methylcellulose or hydroxyethyl cellulose was added, and the mixture was mixed under stirring to give a homogeneous solution. The pH and osmotic pressure were adjusted with hydrochloric acid or sodium hydroxide. The preparations of Comparative Examples were prepared in the same manner as those of the Examples except that GGA was not added.

Each preparation was separately poured, using a glass volumetric pipette, into spray-type nasal drop containers (a container for ROHTO ALGUARD ST Rhinitis Spray (trade name), ROHTO Pharmaceutical Co., Ltd.) each in an amount of 5 mL (5 g) and a spray nozzle was attached to the containers. The nasal drop container was placed so that the spray nozzle was directed parallel to the horizontal plane. At a distance of 50 cm apart from the spray nozzle, Kimtowels wipers (Nippon Paper Crecia Co., Ltd.) were placed. The preparation was then sprayed from the nasal drop container 50 times (see FIG. 4). The Kimtowels wipers were collected and immersed in 100 mL of purified water and the water was stirred. The stirred purified water was transferred, using a glass graduated pipette, into a 96-well plate (flat bottom, made of polystyrene) in an amount of 0.2 mL per well. The absorbance at 450 nm, indicating the amount of uranin, was measured with a microplate reader (VersaMax, Molecular Devices, LLC.) (chamber temperature: 20 to 25° C.). A higher absorbance value at 450 nm indicates a greater amount of the preparation sprayed toward a position 50 cm apart from the spray nozzle.

TABLE 2

| | Comparative | | Comparative | |
|---|---|---|---|---|
| g/100 mL | Example 1 | Example 1 | Example 2 | Example 2 |
| Teprenone | — | 0.05 | — | 0.05 |
| Methylcellulose | 0.125 | 0.125 | — | — |
| Hydroxyethyl | — | — | 0.125 | 0.125 |
| cellulose | | | | |
| Sodium dihydrogen | 0.30 | 0.30 | 0.30 | 0.30 |
| phosphate dihydrate | | | | |
| Disodium hydrogen | 3.20 | 3.20 | 3.20 | 3.20 |
| phosphate docdeca- | | | | |
| hydrate | | | | |
| POE castor oil | 0.02 | 0.02 | 0.02 | 0.02 |
| Polysorbate 80 | 0.50 | 0.50 | 0.50 | 0.50 |
| Uranin | 0.01 | 0.01 | 0.01 | 0.01 |
| Hydrochloric acid | q.s. | q.s. | q.s. | q.s. |
| Sodium hydroxide | q.s. | q.s. | q.s. | q.s. |
| Purified water | q.s. | q.s. | q.s. | q.s. |
| pH | 7.5 | 7.5 | 7.5 | 7.5 |
| Osmotic pressure | 260 | 260 | 260 | 260 |
| (mOsm) | | | | |
| Absorbance | 0.306 | 0.321 | 0.396 | 0.538 |
| (460 nm) | | | | |

Regardless of the type of thickening agent used, the preparations containing GGA apparently showed longer flight distance by spraying from the nasal drop container. When a spray-type nasal drop is applied to the back of the nasal cavity, absorption of the drug is better. Therefore, longer flight distance of the preparation is advantageous for a nasal drop.

(5) Evaluation of the Variability in the Sprayed Amount from Nasal Drop Container

Test preparations whose composition is shown in Table 3 were prepared using teprenone (Wako Pure Chemical industries, Ltd.). Specifically, the test preparations of Examples were prepared as follows. To surfactants (polysorbate 80 and POE castor oil) warmed to 65° C., teprenone was added and dissolved under stirring in hot water bath at 65° C. for 2 minutes. Water at 65° C. was added, a buffer solution containing methylcellulose, hydroxyethyl cellulose, alginic acid, gellan gum, or polyvinylpyrrolidone was added, and the mixture was mixed under stirring to give a homogeneous solution. The pH and osmotic pressure were adjusted with hydrochloric acid or sodium hydroxide. The preparations of Comparative Examples were prepared in the same is manner as those of the Examples except that GGA was not added.

Each preparation was separately poured, using a glass volumetric pipette, into spray-type nasal drop containers (a container for ROHTO ALGUARD ST Rhinitis Spray (trade name), ROHTO Pharmaceutical. Co., Ltd.) each in an amount of 5 mL (5 g) and a spray nozzle was attached to the containers. The preparation was sprayed from the nasal drop container 10 times and the sprayed preparation was collected and weighed.

This test was performed 3 times. After the calculation of the average weight of the 10-time sprayed preparation and its standard deviation, the coefficient of variation (CV) of the weight of the preparation was calculated. The standard deviation was calculated using the STDEVP worksheet function in Microsoft® Excel 2000. The average was calculated using the AVERAGE worksheet function in Microsoft® Excel 2000. The standard deviation was divided by the average to give the CV (coefficient of variation) of the weight of the preparation.

The results are shown in Table 3.

TABLE 3

| | Com- | Com- | Com- | Com- | Com- | | | | | |
|---|---|---|---|---|---|---|---|---|---|---|
| | parative | parative | parative | parative | parative | Example | Example | Example | Example | Example |
| g/100 mL | Example 3 | Example 4 | Example 5 | Example 6 | Example 7 | 3 | 4 | 5 | 6 | 7 |
| Teprenone | — | — | — | — | — | 0.05 | 0.05 | 0.05 | 0.05 | 0.05 |
| Alginic acid | 0.125 | — | — | — | — | 0.125 | — | — | — | — |
| Gellan gum | — | 0.125 | — | — | — | — | 0.125 | — | — | — |
| Methylcellulose | — | — | 0.125 | — | — | — | — | 0.125 | — | — |
| Hydroxyethyl | — | — | — | 0.125 | — | — | — | — | 0.125 | — |
| cellulose | | | | | | | | | | |
| Polyvinyl- | — | — | — | — | 0.125 | — | — | — | — | 0.125 |
| pyrrolidone | | | | | | | | | | |
| Sodium | 0.30 | 0.30 | 0.30 | 0.30 | 0.30 | 0.30 | 0.30 | 0.30 | 0.30 | 0.30 |
| dihydrogen | | | | | | | | | | |
| phosphate | | | | | | | | | | |
| dihydrate | | | | | | | | | | |
| Disodium | 3.20 | 3.20 | 3.20 | 3.20 | 3.20 | 3.20 | 3.20 | 3.20 | 3.20 | 3.20 |
| hydrogen | | | | | | | | | | |
| phosphate | | | | | | | | | | |
| dodecahydrate | | | | | | | | | | |
| POE castor oil | 0.02 | 0.02 | 0.02 | 0.02 | 0.02 | 0.02 | 0.02 | 0.02 | 0.02 | 0.02 |
| Polysorbate 80 | 0.50 | 0.50 | 0.50 | 0.50 | 0.50 | 0.50 | 0.50 | 0.50 | 0.50 | 0.50 |
| Hydrochloric acid | q.s. | q.s. | q.s. | q.s. | q.s. | q.s | q.s. | q.s. | q.s. | q.s. |
| Sodium hydroxide | q.s. | q.s. | q.s. | q.s. | q.s. | q.s. | q.s. | q.s. | q.s. | q.s. |
| Purified water | q.s. | q.s. | q.s. | q.s. | q.s. | q.s. | q.s. | q.s. | q.s. | q.s. |
| pH | 7.5 | 7.5 | 7.5 | 7.5 | 7.5 | 7.5 | 7.5 | 7.5 | 7.5 | 7.5 |
| Osmotic pressure | 260 | 260 | 260 | 260 | 260 | 260 | 260 | 260 | 260 | 260 |
| (mOsm) | | | | | | | | | | |
| CV (Coefficient | 0.149 | 0.040 | 0.062 | 0.116 | 0.588 | 0.032 | 0.025 | 0.050 | 0.083 | 0.365 |
| of variation) | | | | | | | | | | |

When GGA was contained, the variability in the weight of the sprayed preparations was apparently reduced.

(6) Evaluation of the Variability in the Amount of Preparations Dispensed from Eye Drop Container

Test preparations whose composition is shown in Table 4 were prepared using teprenone (Wako Pure Chemical industries, Ltd.). Specifically, the test preparations of Examples were prepared as follows. To surfactants (polysorbate 80 and POE castor oil) warmed to 65° C., teprenone was added and dissolved under stirring in hot water bath at 65° C. for 2 minutes. Water at 65° C. was added, a buffer solution containing hydroxyethyl cellulose, gellan gum, or polyvinylpyrrolidone was added, and the mixture was mixed under stirring to give a homogeneous solution. The pH and osmotic pressure were adjusted with hydrochloric acid or sodium hydroxide. The preparations of Comparative Examples were prepared in the same manner as those of the Examples except that GGA was not added. Each resulting solution was filtered through a membrane filter with a pore size of 0.2 μm (bottle top filter, Thermo Fisher Scientific) to give a clear sterile eye drop.

Each eye drop was separately poured, using a glass volumetric pipette, into eye drop containers (a container for ROHTO Dryaid EX, ROHTO Pharmaceutical Co., Ltd.) each in an amount of 8 mL (8 g). The weight of a single drop of the preparation was measured.

This test was performed 10 times. After the calculation of the average weight of a single drop of the preparation and its standard deviation, the coefficient of variation (CV) of the weight of a single drop of the preparation was calculated. The standard deviation was calculated using the STDEVP worksheet function in Microsoft® Excel 2000. The average was calculated using the AVERAGE worksheet function in Microsoft® Excel 2000. The standard deviation was divided by the average to give the CV (coefficient of variation) of the weight of the preparation.

The results are shown in Table 4.

TABLE 4

| | Comparative | Comparative | Comparative | Example | Example | Example |
|---|---|---|---|---|---|---|
| g/100 mL | Example 8 | Example 9 | Example 10 | 8 | 9 | 10 |
| Teprenone | — | — | — | 0.05 | 0.05 | 0.05 |
| Gellan gum | 0.125 | — | — | 0.125 | — | — |
| Hydroxyethyl | — | 0.125 | — | — | 0.125 | — |
| cellulose | | | | | | |
| Polyvinylpyrrolidone | — | — | 0.125 | — | — | 0.125 |
| Sodium dihydrogen | 0.30 | 0.30 | 0.30 | 0.30 | 0.30 | 0.30 |
| phosphate dihydrate | | | | | | |
| Disodium hydrogen | 3.20 | 3.20 | 3.20 | 3.20 | 3.20 | 3.20 |
| phosphate | | | | | | |
| dodecahydrate | | | | | | |
| POE castor oil | 0.02 | 0.02 | 0.02 | 0.02 | 0.02 | 0.02 |
| Polysorbate 80 | 0.50 | 0.50 | 0.50 | 0.50 | 0.50 | 0.50 |
| Hydrochloric acid | q.s. | q.s | q.s. | q.s. | q.s. | q.s. |
| Sodiuim hydroxide | q.s. | q.s. | q.s. | q.s. | q.s. | q.s. |
| Purified water | q.s. | q.s. | q.s. | q.s. | q.s. | q.s. |
| pH | 7.5 | 7.5 | 7.5 | 7.5 | 7.5 | 7.5 |
| Osmotic pressure | 260 | 260 | 260 | 260 | 260 | 260 |
| (mOsm) | | | | | | |
| CV (Coefficient of | 0.293 | 0.318 | 0.090 | 0.077 | 0.052 | 0.068 |
| variation) | | | | | | |

When GGA was contained, the variability in the weight of a single drop of the preparations was apparently reduced. Usually an eye drop is instilled in an amount of 1 to 3 drops per administration and therefore reduced variability in the amount instilled each time is very advantageous for an eye drop.

### (7) Evaluation of Light Transmission Through Containers

An eye drop containing teprenone was prepared. The composition of the eye drop is shown in Table 5 below. Specifically, the eye drop was prepared as follows. To surfactants (polysorbate 80 and POE castor oil) warmed to 65° C., teprenone was added and dissolved under stirring in hot water bath at 65° C. for 2 minutes. Water at 65° C. was added, a buffer solution was added, and the mixture was mixed under stirring to give a homogeneous solution. The pH and osmotic pressure were adjusted with hydrochloric acid or sodium hydroxide. The resulting solution was filtered through a membrane filter with a pore size of 0.2 μm (bottle top filter, Thermo Fisher Scientific) to give a clear sterile eye drop. Before the preparation of the sterile eye drop, it was confirmed, by HPLC, that adsorption of teprenone to instruments etc., which leads to the loss of the teprenone content, did not occur during the preparation procedure.

The eye drop was separately poured, using a glass volumetric pipette, into 10- to 15-mL plastic or glass containers each in an amount of 5 mL (5 g) and the containers were sealed. These eye drops were placed in the upright position on a test-tube stand and left to stand under direct sunlight for 24 hours (test samples). For the preparation of the controls used for the experiment, an eye drop was prepared and poured into containers in the same manner as the test samples and the thus obtained eye drops were completely covered with aluminum foil to block light (control samples).

The amount of teprenone contained in the test or control eye drops was measured by HPLC and the amount of decomposition (mg/100 mL) by light irradiation was calculated. The amount of decomposition by light irradiation was calculated as follows:

The amount of decomposition by light irradiation (mg/100 mL)=(the amount of teprenone in control samples)−(the amount of teprenone in test samples)

TABLE 5

| g/100 mL | Composition |
|---|---|
| Teprenone | 0.05 |
| Boric acid | 1.30 |
| Borax | 0.40 |
| POE castor oil | 0.02 |
| Polysorbate 80 | 0.50 |
| Hydrochloric acid | q.s. |
| Sodium hydroxide | q.s. |
| Purified water | q.s. |
| Osmotic pressure (mOsm) | 240 |
| pH | 7.5 |

### Measurement of Transmission of Light Through Containers

The transmission of light through the plastic or glass containers was measured.

Specifically, 1 cm×5 cm pieces were cut out from the plastic or glass containers. The cut pieces were used as samples and placed to stand in the sample chamber of a spectrometer (ultraviolet-visible spectrometer UV-2450, Shimadzu Corporation). The transmission of light at each wavelength was measured. The cut pieces from the containers had the area sufficient to cover the light path in the spectrometer.

The conditions of the measurement using the spectrometer are shown below.

### Instrument: UV-2450 (Shimadzu Corporation)

Wavelength range (nm): Start 800, End 200
Scan speed: Medium
Sampling pitch (nm): 1.0
Measurement mode: Single
Type of measurement: Transmission
Slit width (nm): 1.0
Light source switching wavelength (393-282 nm): 282
S/R switching: Normal

The average values of the transmission of light at 245 to 255 nm, 300 to 330 nm, or 350 to 380 nm were calculated to give the average transmission (%). The results are shown in Table 6 and the details of the containers are shown in Table 7.

TABLE 8

| | | Test | Test | Test |
|---|---|---|---|---|
| | | Example 1 | Example 2 | Example 3 |
| Container | | A | B | C |
| Amount of decomposition | | 4.3 | 3.1 | 1.3 |
| (mg/100 mL) | | | | |
| Average | 245-255 (nm) | 37.5 | 0.0 | 0.0 |
| transmission | 300-330 (nm) | 50.7 | 63.1 | 2.2 |
| (%) | 350-380 (nm) | 80.5 | 89.6 | 55.7 |

TABLE 7

| Con- | | | | Model |
|---|---|---|---|---|
| tainer | Material | Volume and type | Manufacturer | number |
| A | Polymethyl- | 15 mL centrifuge | Sumitomo | MS-56150 |
| | terpene | tube | Bakelite | |
| B | Glass | 10 mL centrifuge | IWAKI | 8084CTF10 |
| | | tube | | |
| C | Polyethylene | 15 mL centrifuge | CORNING | 430053 |
| | terephthalate | tube | | |

In the actual situation of commercial distribution, storage or use, the strongest light that would be irradiated on an eye drop solution or nasal drop solution stored in a container is sunlight. This experiment was performed to evaluate to what extent each container can inhibit the decomposition of GGA under sunlight, which has a broad wavelength band.

Under irradiation of direct sunlight, Container C significantly inhibited the decomposition of GGA compared with Containers A and B.

Container C used for the experiment was a colorless container made of polyethylene terephthalate (PET), and its average transmission of 245 to 255 nm light was 35% or less and its average transmission of 300 to 330 nm light was 50% or less. Container A used for the experiment was a colorless container made of polymethylpentene, and its average transmission of 245 to 255 nm light was over 35% and its average transmission of 300 to 330 nm light was over 50%. Container B used for the experiment was a colorless container made of glass, and its average transmission of 245 to 255 nm light was 35% or less but its average transmission of 300 to 330 nm light was over 50%.

Therefore, when the mucosally applied preparation comprising GGA is stored in a container having a part with an average transmission of 245 to 255 nm light of 35% or less or an average transmission of 300 to 330 nm light of 50% or less, the decomposition of GGA at the time of actual commercial distribution, storage, and use is efficiently inhibited.

In addition, in terms of the average transmission of light with a wavelength range other than the above two ranges, for example, the average transmission of light with a wavelength range of 350 to 380 nm, there was not much difference among Containers A, B and C. Therefore, it is understood that the average transmission of light with wavelength ranges of 300 to 330 nm and 245 to 255 nm is important for the inhibition of the decomposition of GGA.

### INDUSTRIAL APPLICABILITY

The preparation of the present invention is used as a prophylactic, ameliorating or therapeutic drug for a retinal disease, which is generally serious, and is administered to the mucosa so that systemic circulation of the preparation is prevented. Therefore, the preparation of the present invention is an excellent preparation preferred by a patient.

1. A method for preventing, ameliorating or treating a retinal disease, the method comprising the step of applying geranylgeranylacetone to the mucosa of a patient with a retinal disease.

2. The method according to claim 1, wherein the mucosa is eye mucosa, nasal mucosa, oral cavity mucosa or pharyngeal mucosa.

3. The method according to claim 1, comprising applying a pharmaceutical preparation comprising geranylgeranylacetone to the mucosa of a patient with a retinal disease, the pharmaceutical preparation being an eye drop, an eye wash, a contact lens-wearing solution, an eye ointment, a nasal drop, a nasal douche, a nasal ointment, an ear drop, an ear ointment, an oropharyngeal ointment, a sublingual tablet, a buccal preparation, an oral-mucosa patch or an inhalant.

4. The method according to claim 1, wherein the retinal disease is at least one disease selected from the group consisting of glaucoma, retinitis pigmentosa, age-related macular degeneration, diabetic retinopathy, retinal detachment, diabetic maculopathy, hypertensive retinopathy, retinal vascular occlusion, retinal arteriosclerosis, retinal tear, retinal hole, macular hole, ophthalmorrhagia, posterior vitreous detachment, pigmented paravenous retinochoroidal atrophy, gyrate atrophy of the retina and choroid, choroideremia, crystalline retinopathy, retinitis punctata albescens, cone dystrophy, central areolar choroidal dystrophy, Doyne's honeycomb retinal dystrophy, vitelliform macular dystrophy, cystoid macular edema, occult macular dystrophy, Stargardt disease, retinoschisis, central serous chorioretinopathy, spinocerebellar ataxia type 7, familial exudative vitreoretinopathy, enhanced S-cone syndrome, angioid streaks, autosomal dominant optic atrophy, autosomal dominant drusen, acute zonal occult outer retinopathy, cancer-associated retinopathy, light damage, and ischemic retinopathy.

5. The method according to claim 1, comprising applying, to the mucosa of a patient with a retinal disease, a pharmaceutical preparation comprising 0.00001 to 10% by weight of geranylgeranylacetone relative to the total amount of the pharmaceutical preparation.

6. The method according to claim 1, comprising applying an aqueous or oily composition comprising geranylgeranylacetone to the mucosa of a patient with a retinal disease.

7. The method according to claim 1, comprising applying, to the mucosa of a patient with a retinal disease, a geranylgeranylacetone-containing composition in the form of a liquid, a fluid, a gel, a semisolid or a solid.

8. The method according to claim 1, comprising applying geranylgeranylacetone stored in a container to the mucosa of a patient with a retinal disease.

9. The method according to claim 8, comprising applying, to the mucosa of a patient with a retinal disease, geranylgeranylacetone stored in a container having a part which satisfies the following conditions (a) and/or (b):
(a) average transmission of light with a wavelength of 245 to 255 nm is 35% or less;
(b) average transmission of light with a wavelength of 300 to 330 nm is 50% or less.

10. The method according to claim 9, comprising applying, to the mucosa of a patient with a retinal disease, geranylgeranylacetone stored in a container having a part which satisfies the above conditions (a) and (b).

11. The method according to claim 1, wherein the daily dose of GGA is 1 ng to 2000 mg.

12. A method for protecting a retinal cell, the method comprising the step of applying geranylgeranylacetone to the mucosa of a patient with a retinal disease.

13. The method according to claim 12, wherein the mucosa is eye mucosa, nasal, mucosa, oral cavity mucosa or pharyngeal mucosa.

14. The method according to claim 13, comprising applying, to the mucosa of a patient with a retinal disease, a pharmaceutical preparation comprising 0.00001 to 10% by weight of geranylgeranylacetone relative to the total amount of the pharmaceutical preparation.

15. The method according to claim 13, wherein the daily dose of GGA is 1 ng to 2000 mg.

16. A method for inhibiting the degeneration, impairment or destruction of a retinal cell, the method comprising the step of applying geranylgeranylacetone to the mucosa of a patient with a retinal disease.

17. The method according to claim 16, wherein the mucosa is eye mucosa, nasal mucosa, oral cavity mucosa or pharyngeal mucosa.

18. The method according to claim 16, comprising applying, to the mucosa of a patient with a retinal disease, a pharmaceutical preparation comprising 0.00001 to 10% by weight of geranylgeranylacetone relative to the total amount of the pharmaceutical preparation.

19. The method according to claim 16, wherein the daily dose of GGA is 1 ng to 2000 mg.
